(19) United States
(12) Patent Application Publication
(10) Pub. No.: US 2007/0020216 A1
(43) Pub. Date: Jan. 25, 2007

(54) Compositions for Treating Keratinous Surfaces

(75) Inventors: Gale McElroy Reinhart, Middletown, NJ (US); Julio Gans Russ, Westfield, NJ (US)

Correspondence Address: JULIE BLACKBURN;REVLON CONSUMER PRODUCTS CORPORATION, 237 PARK AVENUE, NEW YORK, NY, 10017, US

(73) Assignee: Revlon Consumer Products Corporation

(21) Appl. No.: 11/420,362

(22) Filed: May 25, 2006

Related U.S. Application Data
(60) Provisional application No. 60/702,541, filed on Jul. 25, 2005.

(51) Int. Cl. A61K 8/64

(52) U.S. Cl. 424/70.7; 424/70.14

(57) ABSTRACT

A cosmetic composition comprising a hexapeptide having Sequence ID No. 1 in a cosmetically acceptable carrier, and use of such cosmetic compositions in improving skin conditions associated with aging such as winkles, fine lines, laxity, mottled pigmentation, and sallowness.

### RELATED APPLICATION

This application claims priority to provisional patent application Ser. No. 60/702,541, filed Jul. 25, 2005.

### TECHNICAL FIELD

The invention is in the field of compositions for treatment of keratinous surfaces such as skin (including lips), hair and nails.

### BACKGROUND OF THE INVENTION

Cosmetics companies are on an eternal quest to provide cosmetics that provide an immediate as well as long term beauty benefit. For example, women typically use foundation makeup to cover skin imperfections and improve the appearance of facial skin, not thinking of foundation as a skin conditioning composition. The same is true for other color cosmetic products such as lipsticks, blushes, concealers, eyeshadows, and the like. Typically they are used by women to provide immediate beauty benefits, and are not considered by such users to be skin conditioning or anti-aging products. Products such as skin creams and lotions are often thought of as providing long term beauty benefit in that consistent use of such products over a longer time period will provide some benefit such as wrinkle reduction, improvement in skin tone, and so on, with respect to the keratinous surface to which they are applied. As the baby boomer population ages, a much larger percentage of women must contend with the effects of age on skin. Wrinkles, sags, age spots, and other effects of age become evident. Such consumers have a need for skin treatment and color products that provide both skin conditioning and anti-aging properties as well as the desired immediate beauty benefit.

It has been discovered that a certain hexapeptide provides excellent anti-aging properties and is compatible with a wide variety of the ingredients used in cosmetic products.

It is an object of the invention to provide skin conditioning and beautifying compositions containing anti-aging and skin conditioning hexapeptides.

It is a further object of the invention to provide color cosmetic compositions comprising hexapeptides.

It is a further object of the invention to provide cosmetic compositions comprising a hexapeptide having a certain amino acid sequence.

### SUMMARY OF THE INVENTION

The invention is directed to a cosmetic composition comprising a hexapeptide having an amino acid sequence (Sequence ID No. 1):

serine-isoleucine-lysine-valine-alanine-valine

The invention is further directed to a cosmetic composition for improving skin conditions associated with aging such as wrinkles, fine lines, laxity, mottled pigmentation, and sallowness comprising a hexapeptide having Sequence ID No. 1 in a cosmetically acceptable carrier.

The invention is further directed to a color cosmetic composition comprising at least one hexapeptide having Sequence ID No. 1 and at least one cosmetically acceptable pigment or powder.

The invention is further directed to a water and oil emulsion color cosmetic composition comprising a hexapeptide having Sequence ID No. 1.

The invention is further directed to pigmented anhydrous cosmetic composition comprising a hexapeptide having Sequence ID No. 1.

### DETAILED DESCRIPTION

The term “keratinous surfaces” means the surfaces of skin, hair and nails. The term “skin” when used herein is in the broad sense meaning the skin of the face, body, and neck as well as the lips.

The term “skin benefit agent” means an agent that is used on skin to provide certain benefits. Skin benefit agents include particulate sunscreens, UVA or UVB chemical sunscreens, botanical extracts, vitamins or antioxidants, other peptides, humectants, all as identified herein and in the same percentage ranges.

The compositions of the invention may be anhydrous, or in the emulsion, solution, or suspension form. If in the emulsion form, the emulsions may be water-in-oil or oil-in-water. Suitable water and oil emulsions contain about 0.1-95%, preferably about 0.5-85%, more preferably about 5-85% by weight of the total composition of water and about 0.1 -99%, preferably about 1-90%, more preferably about 3-85% by weight of the total composition of oil.

I. The Hexapeptide

The hexapeptide used in the compositions of the invention has an amino acid sequence as set forth below (Sequence ID No. 1):

serine-isoleucine-lysine-valine-alanine-valine The peptide may be purchased from Lipotec under the tradename Serilesine™, and has the C.T.F.A. name Hexapeptide-10.

The compositions of the invention preferably contain from about 0.00001-25%, preferably about 0.00005-20%, more preferably about 0.001-18% by weight of the total composition of a hexapeptide having Sequence ID No. 1.

II. The Cosmetically Acceptable Carrier

The hexapeptide having Sequence ID No. 1 may be incorporated into a variety of skin care compositions, including but not limited to gels, creams, lotions, sunscreens, and the like. In addition, the hexapeptide used in the compositions of the invention may be used in color cosmetic compositions such as foundation makeups, blushes, eyeshadows, mascaras, concealers, eyeliners, lip colors, nail colors, and so on.

Compositions that may be found in the emulsion form, for example, creams, lotions, sunscreens, foundation makeups, concealers, lipcolor, and the like, may be water-in-oil or oil-in-water emulsions. Preferably such emulsions comprise from about 0.1-95%, preferably about 0.5-85%, more preferably about 5-85% by weight of the total composition of water and about 0.1 -99%, preferably about 1-90%, more preferably about 3-85% by weight of the total composition of oil. In addition to oil, the other ingredients that may be found in such compositions include surfactants, sunscreens, particulates, film forming polymers, humectants, thickeners, structuring agents, and so on.

Other compositions in accordance with the invention, for example, eyeshadows, blushes, some types of concealers, lipcolor, some types of lashcolor, may be found in the anhydrous form. Typically such compositions comprise an oily phase ranging from about 0.1-99%, preferably about 1-90%, more preferably about 3-85% by weight of the total composition, with particulates, pigments, and other ingredients as further identified below.

A. Oils

If present, suggested ranges for such oils in the compositions of the invention are about 0.1-90%, preferably 0.5-75%, more preferably 1-60% by weight of the total composition. The oils used may be volatile or nonvolatile, and are liquid at room temperature. The term “volatile” means that the oil has a measurable vapor pressure, or a vapor pressure of at least about 2 mm. of mercury at 20° C. The term “nonvolatile” means that the oil has a vapor pressure of less than about 2 mm. of mercury at 20° C.

1. Volatile Oils

Suitable volatile oils generally have a viscosity of about 0.5 to 10 centipoise at 25° C. Suitable volatile oils include linear silicones, cyclic silicones, paraffinic hydrocarbons, or mixtures thereof.

Cyclic silicones (or cyclomethicones) are of the general formula: 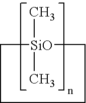
where n=3-6.

Linear volatile silicones in accordance with the invention have the general formula:
(CH₃)₃Si—O—[Si(CH₃)₂—O]ₙ—Si(CH₃)₃
where n=0-5, preferably 0-4.

Linear and cyclic volatile silicones are available from various commercial sources including Dow Corning Corporation and General Electric. The Dow Corning volatile silicones are sold under the tradenames Dow Corning 244, 245, 344, and 200 fluids. These fluids comprise octamethylcyclotetrasiloxane, decamethylcyclopentasiloxane, dodecamethylcyclohexasiloxane and the like. Also suitable are linear volatile silicones such as hexamethyldisiloxane, octamethyltrisiloxane, decamethyltetrasiloxane, dodecamethylpentasiloxane, and mixtures thereof.

Also suitable as the volatile oils are various straight or branched chain paraffinic hydrocarbons having 5 to 20 carbon atoms, more preferably 8-16 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference. Preferred volatile paraffinic hydrocarbons have a molecular weight of 70-225, preferably 160 to 190 and a boiling point range of 30 to 320, preferably 60-260 degrees C., and a viscosity of less than 10 cs. at 25 degrees C. Such paraffinic ydrocarbons are available from EXXON under the ISOPARS trademark, and from the Permethyl Corporation. Suitable C₁₂ isoparaffins are manufactured by Permethyl Corporation under the tradename Permethyl 99A. Various C₁₆ isoparaffins commercially available, such as isohexadecane (having the tradename Permethyl R), are also suitable. Transfer resistant cosmetic sticks of the invention will generally comprise a mixture of volatile silicones and volatile paraffinic hydrocarbons.

2. Non-Volatile Oils

A wide variety of nonvolatile oils are also suitable for use in the cosmetic compositions of the invention. The nonvolatile oils generally have a viscosity of greater than about 5 to 10 centipoise at 25° C., and may range in viscosity up to about 1,000,000 centipoise at 25° C.

(a). Esters

Suitable esters are mono-, di-, and triesters. The composition may comprise one or more esters selected from the group, or mixtures thereof.

(i). Monoesters

Monoesters are defined as esters formed by the reaction of a monocarboxylic acid having the formula R—COOH, wherein R is a straight or branched chain saturated or unsaturated alkyl having 2 to 30 carbon atoms, or phenyl; and an alcohol having the formula R—OH wherein R is a straight or branched chain saturated or unsaturated alkyl having 2-30 carbon atoms, or phenyl. Both the alcohol and the acid may be substituted with one or more hydroxyl groups, Either one or both of the acid or alcohol may be a “fatty” acid or alcohol, and may have from about 6 to 30 carbon atoms. Examples of monoester oils that may be used in the compositions of the invention include hexyldecyl benzoate, hexyl laurate, hexadecyl isostearate, hexydecyl laurate, hexyldecyl octanoate, hexyldecyl oleate, hexyldecyl palmitate, hexyldecyl stearate, hexyldodecyl salicylate, hexyl isostearate, butyl acetate, butyl isostearate, butyl oleate, butyl octyl oleate, cetyl palmitate, cetyl octanoate, cetyl laurate, cetyl lactate, isostearyl isononanoate, cetyl isononanoate, cetyl stearate, stearyl lactate, stearyl octanoate, stearyl heptanoate, stearyl stearate, and so on.

(ii). Diesters

Suitable diesters are the reaction product of a dicarboxylic acid and an aliphatic or aromatic alcohol or an aliphatic or aromatic alcohol having at least two substituted hydroxyl groups and a monocarboxylic acid. The dicarboxylic acid may contain from 2 to 30 carbon atoms, and may be in the straight or branched chain, saturated or unsaturated form. The dicarboxylic acid may be substituted with one or more hydroxyl groups. The aliphatic or aromatic alcohol may also contain 2 to 30 carbon atoms, and may be in the straight or branched chain, saturated, or unsaturated form. Preferably, one or more of the acid or alcohol is a fatty acid or alcohol, i.e. contains 14-22 carbon atoms. The dicarboxylic acid may also be an alpha hydroxy acid. Examples of diester oils that may be used in the compositions of the invention include diisostearyl malate, neopentyl glycol dioctanoate, dibutyl sebacate, di-C₁₂-₁₃ alkyl malate, dicetearyl dimer dilinoleate, dicetyl adipate, diisocetyl adipate, diisononyl adipate, diisostearyl dimer dilinoleate, diisostearyl fumarate, diisostearyl malate, and so on.

(iii). Triesters

Suitable triesters comprise the reaction product of a tricarboxylic acid and an aliphatic or aromatic alcohol or alternatively the reaction product of an aliphatic or aromatic alcohol having three or more substituted hydroxyl groups with a monocarboxylic acid. As with the mono- and diesters mentioned above, the acid and alcohol contain 2 to 30 carbon atoms, and may be saturated or unsaturated, straight or branched chain, and may be substituted with one or more hydroxyl groups. Preferably, one or more of the acid or alcohol is a fatty acid or alcohol containing 14 to 22 carbon atoms. Examples of triesters include triarachidin, tributyl citrate, triisostearyl citrate, tri C₁₂₋₁₃ alkyl citrate, tricaprylin, tricaprylyl citrate, tridecyl behenate. trioctyldodecyl citrate, tridecyl behenate, tridecyl cocoate, tridecyl isononanoate, and so on.

Esters suitable for use in the composition are further described on pages 1670-1676 of the C.T.F.A. Cosmetic ingredient Dictionary and Handbook, Eighth Edition, 2000, which is hereby incorporated by reference in its entirety.

(b). Hydrocarbon Oils

It may be desirable to incorporate one or more non-volatile hydrocarbon oils into the composition. The term “nonvolatile” means that the oil has a vapor pressure of less than about 2 mm. of mercury at 20° C.

Suitable nonvolatile hydrocarbon oils include paraffinic hydrocarbons and olefins, preferably those having greater than 20 carbon atoms. Examples of such hydrocarbon oils include C₂₄₋₂₈ olefins, C₃₀₋₄₅ olefins, C₂₀₋₄₀ isoparaffins, hydrogenated polyisobutene, polyisobutene, mineral oil, pentahydrosqualene, squalene, squalane, and mixtures thereof.

(c). Lanolin Oil

Also suitable for use in the composition is lanolin oil or derivatives thereof containing hydroxyl, alkyl, or acetyl groups, such as hydroxylated lanolin, isobutylated lanolin oil, acetylated lanolin, acetylated lanolin alcohol, and so on.

(d). Glyceryl Esters of Fatty Acids

Naturally occurring glyceryl esters of fatty acids, or triglycerides, are also suitable for use in the compositions. Both vegetable and animal sources may be used. Examples of such oils include castor oil, lanolin oil, C₁₀₋₁₈ triglycerides, caprylic/capric/triglycerides, coconut oil, corn oil, cottonseed oil, linseed oil, mink oil, olive oil, palm oil, illipe butter, rapeseed oil, soybean oil, sunflower seed oil, walnut oil, and the like.

Also suitable are synthetic or semi-synthetic glyceryl esters, e.g. fatty acid mono-, di-, and triglycerides which are natural fats or oils that have been modified, for example, acetylated castor oil, or mono-, di- or triesters of polyols such as glyceryl stearate, diglyceryl diiosostearate, polyglycera,-4 isostearate, polyglyceryl-6 ricinoleate, glyceryl dioleate, glyceryl diisotearate, glyceryl trioctanoate, diglyceryl distearate, glyceryl linoleate, glyceryl myristate, glyceryl isostearate, PEG castor oils, PEG glyceryl oleates, PEG glyceryl stearates, PEG glyceryl tallowates, and so on.

(e). Nonvolatile Silicones

Nonvolatile silicone oils, both water soluble and water insoluble, are also suitable for use in the composition. Such silicones preferably have a viscosity ranging from about 10 to 600,000 centistokes, preferably 20 to 100,000 centistokes at 25° C. Suitable water insoluble silicones include amine functional silicones such as amodimethicone; phenyl substituted silicones such as bisphenylbexamethicone, phenyl trimethicone, or polyphenylmethylsiloxane; dimethicone, alkyl substituted dimethicones, and mixtures thereof.

Such silicones have the following general formula: 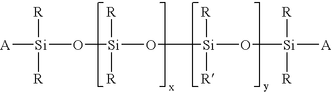
wherein R and R′ are each independently C₁₋₃₀ alkyl, phenyl or aryl, trialkylsiloxy, and x and y are each independently 0-1,000,000; with the proviso that there is at least one of either x or y, and A is siloxy endcap unit. Preferred is where A is a methyl siloxy endcap unit, in particular trimethylsiloxy, and R and R′ are each independently a C₁₋₃₀ straight or branched chain alkyl, phenyl, or trimethylsiloxy, more preferably a C₁₋₂₂ alkyl, phenyl, or trimethylsiloxy, most preferably methyl, phenyl, or trimethylsiloxy, and resulting silicone is dimethicone, phenyl dimethicone, diphenyl dimethicone, or phenyl trimethicone. Other examples include alkyl dimethicones such as cetyl dimethicone, and the like wherein at least one R is a fatty alkyl (C₁₂, C₁₄, C₁₆, C₁₈, or C₂₂), and the other R is methyl, and A is a trimethylsiloxy endcap unit.

(f). Fluorinated Oils

Various types of fluorinated oils may also be suitable for use in the compositions including but not limited to fluorinated silicones, fluorinated esters, or perfluropolyethers. Particularly suitable are fluorosilicones such as trimethylsilyl endcapped fluorosilicone oil, polytrifluoropropylmethylsiloxanes, and similar silicones such as those disclosed in U.S. Pat. No. 5,118,496 which is hereby incorporated by reference, Perfluoropolyethers include those disclosed in U.S. Pat. Nos. 5,183,589, 4,803,067, 5,183,588 all of which are hereby incorporated by reference, which are commercially available from Montefluos under the trademark Fomblin.

Fluoroguerbet esters are also suitable oils. The term “guerbet ester” means an ester which is formed by the reaction of a guerbet alcohol having the general formula: 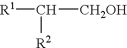
and a fluoroalcohol having the following general formula:
CF₃—(CF₂)ₙ—CH₂—CH₂—OH
wherein n is from 3 to 40,
with a carboxylic acid having the general formula:
R₃—COOH, or
HOOC—R³—COOH
wherein R¹, R², and R³ are each independently a straight or branched chain alkyl.

The guerbet ester may be a fluoro-guerbet ester, which is formed by the reaction of a guerbet alcohol and carboxylic acid (as defined above), and a fluoroalcohol having the following general formula:
CF₃-(CF₂)ₙ—CH₂—CH₂—OH
wherein n is from 3 to 40.

Examples of suitable fluoro guerbet esters are set forth in U.S. Pat. No. 5,488,121 which is hereby incorporated by reference. Suitable fluoro-guerbet esters are also set forth in U.S. Pat. No. 5,312,968 which is hereby incorporated by reference. One type of such an ester is fluorooctyldodecyl meadowfoamate, sold under the tradename Silube GME-F by Siltech, Norcross, Ga.

B. Surfactants

The compositions of the invention may comprise about 0.01-20%, preferably about 0.1-15%, more preferably about 0.5-10% by weight of the total composition of one or more surfactants. The surfactants present may be anionic, nonionic, cationic, zwitterionic, or amphoteric.

1. Nonionic Surfactants

(a) Organic Nonionic Surfactants

The composition may comprise one or more nonionic organic surfactants. Suitable nonionic surfactants include alkoxylated alcohols, or ethers, formed by the reaction of an alcohol with an alkylene oxide, usually ethylene or propylene oxide. Preferably the alcohol is either a fatty alcohol having 6 to 30 carbon atoms Examples of such ingredients include Steareth 2-100, which is formed by the reaction of stearyl alcohol and ethylene oxide and the number of ethylene oxide units ranges from 2 to 100; Beheneth 5-30 which is formed by the reaction of behenyl alcohol and ethylene oxide where the number of repeating ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on.

Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol.

Also suitable as nonionic surfactants are carboxylic acids, which are formed by the reaction of a carboxylic acid with an alkylene oxide or with a polymeric ether. The resulting products have the general formula: 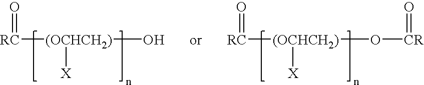
where RCO is the carboxylic ester radical, X is hydrogen or lower alkyl, and n is the number of polymerized alkoxy groups. In the case of the diesters, the two RCO-groups do not need to be identical. Preferably, R is a C₆₋₃₀ straight or branched chain, saturated or unsaturated alkyl, and n is from 1-100.

Monomeric, homopolymeric, or block copolymeric ethers are also suitable as nonionic surfactants. Typically, such ethers are formed by the polymerization of monomeric alkylene oxides, generally ethylene or propylene oxide. Such polymeric ethers have the following general formula: 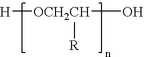
wherein R is H or lower alkyl and n is the number of repeating monomer units, and ranges from 1 to 500.

Other suitable nonionic surfactants include alkoxylated sorbitan and alkoxylated sorbitan derivatives. For example, alkoxylation, in particular ethoxylation of sorbitan provides polyalkoxylated sorbitan derivatives. Esterification of polyalkoxylated sorbitan provides sorbitan esters such as the polysorbates. Examples of such ingredients include Polysorbates 20-85, sorbitan oleate, sorbitan palmitate, sorbitan sesquiisostearate, sorbitan stearate, and so on.

(b). Silicone Surfactants

Also suitable as nonionic surfactants are various types of silicone surfactants, which are defined as silicone polymers that have at least one hydrophilic radical and at least one lipophilic radical. These silicone surfactants may be liquids or solids at room temperature. The silicone surfactant is, generally, a water-in-oil or oil-in-water type surfactant having a Hydrophile/Lipophile Balance (HLB) ranging from about 2 to 18. Preferably the silicone surfactant is a nonionic surfactant having an HLB ranging from about 2 to 12, preferably about 2 to 10, most preferably about 4 to 6. The HLB of a nonionic surfactant is the balance between the hydrophilic and lipophilic portions of the surfactant and is calculated according to the following formula:
HLB=7+11.7×log M_{w}/Mₒ
where M_{w} is the molecular weight of the hydrophilic group portion and Mₒ is the molecular weight of the lipophilic group portion.

The term “silicone surfactant” means an organosiloxane polymer containing a polymeric backbone including repeating siloxy units that may have cyclic, linear or branched repeating units, e.g. di(lower)alkylsiloxy units, preferably dimethylsiloxy units. The hydrophilic portion of the organosiloxane is generally achieved by substitution onto the polymeric backbone of a radical that confers hydrophilic properties to a portion of the molecule. The hydrophilic radical may be substituted on a terminus of the polymeric organosiloxane, or on any one or more repeating units of the polymer. In general, the repeating dimethylsiloxy units of modified polydimethylsiloxane emulsifiers are lipophilic in nature due to the methyl groups, and confer lipophilicity to the molecule. In addition, longer chain alkyl radicals, hydroxy-polypropyleneoxy radicals, or other types of lipophilic radicals may be substituted onto the siloxy backbone to confer further lipophilicity and organocompatibility. If the lipophilic portion of the molecule is due in whole or part to a specific radical, this lipophilic radical may be substituted on a terminus of the organosilicone polymer, or on any one or more repeating units of the polymer. It should also be understood that the organosiloxane polymer in accordance with the invention should have at least one hydrophilic portion and one lipophilic portion.

The term “hydrophilic radical” means a radical that, when substituted onto the organosiloxane polymer backbone, confers hydrophilic properties to the substituted portion of the polymer. Examples of radicals that will confer hydrophilicity are hydroxy-polyethyleneoxy, hydroxyl, carboxylates, and mixtures thereof.

The term “lipophilic radical” means an organic radical that, when substituted onto the organosiloxane polymer backbone, confers lipophilic properties to the substituted portion of the polymer. Examples of organic radicals that will confer lipophilicity are C₁₋₄₀ straight or branched chain alkyl, fluoro, aryl, aryloxy, C₁₋₄₀ hydrocarbyl acyl, hydroxy-polypropyleneoxy, or mixtures thereof. The C₁₋₄₀ alkyl may be non-interrupted, or interrupted by one or more oxygen atoms, a benzene ring, amides, esters, or other functional groups.

The polymeric organosiloxane surfactant used in the invention may have any of the following general formulas:
MₓQ_{y,} or
MₓT_{y,} or
MDₓD′_{y}D″_{z}M
wherein each M is independently a substituted or unsubstituted trimethylsiloxy endcap unit. If substituted, one or more of the hydrogens on the endcap methyl groups are substituted, or one or more methyl groups are substituted with a substituent that is a lipophilic radical, a hydrophilic radical, or mixtures thereof. T is a trifunctional siloxy unit having the empirical formula RSiO_{1.5} or R′SiO_{1.5} wherein R is methyl and R′ is a C₂₋₂₂ alkyl or phenyl, Q is a quadrifunctional siloxy unit having the empirical formula SiO₂, and D, D′, D″, x, y, and z are as set forth below, with the proviso that the compound contains at least one hydrophilic radical and at least one lipophilic radical. Preferred is a linear silicone of the formula.
MDₓD′_{y}D″_{z}M
wherein M=RRRSiO_{0.5}
D=RRSiO_{1.0}
D′=RR′SiO_{1.0}
D″=R′R′SiO_{1.0}
x, y, and z are each independently 0-1000,
where R is methyl or hydrogen, and R′ is a hydrophilic radical or a lipophilic radical,
with the proviso that the compound contains at least one hydrophilic radical and at least one lipophilic radical.
   Most preferred is wherein:
M=trimethylsiloxy
D=Si[(CH₃)][(CHR₂)ₙCH₃]O_{1.0} where n=0-40,
D′=Si [(CH₃)][(CH₂)ₒ—O—PE)]O_{1.0} where PE is (—C₂H₄O)ₐ(—C₃H₆O)_{b}H, o=0-40,
a=1-100 and b=1-100, and
D″=Si (CH₃)₂O_{1.0}

More specifically, suitable silicone surfactants have the formula: 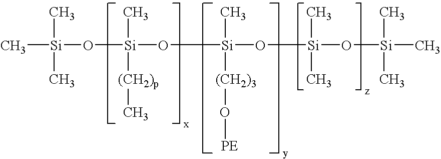
wherein p is 0-40, and
PE is (—C₂H₄O)ₐ(—C₃H₆O)_{b}—H
   where x, y, z, a, and b are such that the maximum molecular weight of the polymer is approximately about 50,000.

Another type of silicone surfactant suitable for use in the compositions of the invention are emulsifiers sold by Union Carbide under the Silwet™ trademark. These surfactants are represented by the following generic formulas:
(Me₃Si)_{y-2}[(OSiMe₂)_{x/y}O—PE]_{y}
wherein PE is
—(EO)ₘ(PO)ₙR
where R=lower alkyl or hydrogen
Me=methyl
EO is polyethyleneoxy
PO is polypropyleneoxy
m and n are each independently 1-5000
x and y are each independently 0-5000, and 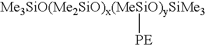
   wherein PE is
   —CH₂CH₂CH₂O(EO)ₘ(PO)ₙZ
where Z=lower alkyl or hydrogen, and
Me, m, n, x, y, EO and PO are as described above,
   with the proviso that the molecule contains a lipophilic portion and a hydrophilic portions Again, the lipophilic portion can be supplied by a sufficient number of methyl groups on the polymer.

As with both types of silicone surfactants, the hydrophilic radical can be substituted on the terminal portions of the silicone, or in other words in the alpha or omega positions or both.

Also suitable as nonionic silicone surfactants are hydroxy-substituted silicones such as dimethiconol, which is defined as a dimethyl silicone substituted with terminal hydroxy groups.

Examples of silicone surfactants are those sold by Dow Corning under the tradename Dow Corning 3225C Formulation Aid, Dow Coming 190 Surfactant, Dow Corning 193 Surfactant, Dow Corning Q2-5200, Abil WE97, and the like are also suitable. In addition, surfactants sold under the tradename Silwet by Union Carbide, and surfactants sold by Troy Corporation under the Troysol tradename, those sold by Taiwan Surfactant Co. under the tradename Ablusoft, those sold by Hoechst under the tradename Arkophob, are also suitable for use in the invention.

2. Anionic Surfactants

If desired the composition may contain one or more anionic surfactants. If so, suggested ranges of anionic surfactant range from about 0.01-25%, preferably 0.5-20%, more preferably about 1-15% by weight of the total composition. Suitable anionic surfactants include alkyl and alkyl ether sulfates generally having the formula ROSO₃M and RO(C₂H₄O)ₓSO₃M wherein R is alkyl or alkenyl of from about 10 to 20 carbon atoms, x is 1 to about 10 and M is a water soluble cation such as ammonium, sodium, potassium, or triethanolamine cation.

Another type of anionic surfactant which may be used in the compositions of the invention are water soluble salts of organic, sulfuric acid reaction products of the general formula:
R₁—SO₃—M
wherein R₁ is a straight or branched chain, saturated aliphatic hydrocarbon radical having from about 8 to about 24 carbon atoms, preferably 12 to about 18 carbon atoms; and M is a cation. Examples of such anionic surfactants are salts of organic sulfuric acid reaction products of hydrocarbons such as n-paraffins having 8 to 24 carbon atoms, and a sulfonating agent, such as sulfur trioxide.

Also suitable as anionic surfactants are reaction products of fatty acids esterified with isethionic acid and neutralized with sodium hydroxide, or fatty acids reacted with alkanolamines or ammonium hydroxides. The fatty acids may be derived from coconut oil, for example. Examples of fatty acids also include lauric acid, stearic acid, oleic acid, palmitic acid, and so on.

In addition, succinates and succinimates are suitable anionic surfactants. This class includes compounds such as disodium N-octadecylsulfosuccinate; tetrasodium N-(1,2-dicarboxyethyl)-N-octadecylsulfosuccinate; and esters of sodium sulfosuccinic acid e.g. the dihexyl ester of sodium sulfosuccinic acid, the dioctyl ester of sodium sulfosuccinic acid, and the like.

Other suitable anionic surfactants include olefin sulfonates having about 12 to 24 carbon atoms. The term “olefin sulfonate” means a compound that can be produced by sulfonation of an alpha olefin by means of uncomplexed sulfur trioxide, followed by neutralization of the acid reaction mixture in conditions such that any sultones, which have been formed in the reaction are hydrolyzed to give the corresponding hydroxy-alkanesulfonates. The alpha olefin from which the olefin sulfonate is derived is a mono-olefin having about 12 to 24 carbon atoms, preferably about 14 to 16 carbon atoms.

Other classes of suitable anionic organic surfactants are the beta-alkoxy alkane sulfonates or water soluble soaps thereof, such as the salts of C₁₀₋₂₀ fatty acids, for example coconut and tallow based soaps. Preferred salts are ammonium, potassium, and sodium salts.

Still another class of anionic surfactants include N-acyl amino acid surfactants and salts thereof (alkali, alkaline earth, and ammonium salts) having the formula: 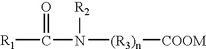
wherein R₁ is a C₈₋₂₄ alkyl or alkenyl radical, preferably C₁₀₋₁₈; R₂ is H, C₁₋₄ alkyl, phenyl, or —CH₂COOM: R₃ is CX₂— or C₁₋₂ alkoxy, wherein each X independently is H or a C₁₋₆ alkyl or alkylester, n is from 1 to 4. and M is H or a salt forming cation as described above. Examples of such surfactants are the N-acyl sarcosinates, including lauroyl sarcosinate, myristoyl sarcosinate, cocoyl sarcosinate, and oleoyl sarcosinate, preferably in sodium or potassium forms.

3. Cationic Zwitterionic or Betaine Surfactants

Certain types of amphoteric, zwitterionic, or cationic surfactants may also be used in the compositions. Descriptions of such surfactants are set forth in U.S. Pat. No. 5,843,193, which is hereby incorporated by reference in its entirety.

Amphoteric surfactants that can be used in the compositions of the invention are generally described as derivatives of aliphatic secondary or tertiary amines wherein one aliphatic radical is a straight or branched chain alkyl of 8 to 18 carbon atoms and the other aliphatic radical contains an anionic group such as carboxy, sulfonate, sulfate, phosphate, or phosphonate.

Suitable amphoteric surfactants may be imidazolinium compounds having the general formula: 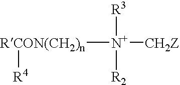
wherein R¹ is C₈₋₂₂ alkyl or alkenyl, preferably C₁₂₋₁₆; R² is hydrogen or CH₂CO₂M, R³ is CH₂CH₂OH or CH₂CH₂OCH₂CHCOOM; R⁴ is hydrogen, CH₂CH₂OH, or CH₂CH₂OCH₂CH₂COOM, Z is CO₂M or CH₂CO₂M, n is 2 or 3, preferably 2, M is hydrogen or a cation such as an alkali metal, alkaline earth metal, ammonium, or alkanol ammonium cation. Examples of such materials are marketed under the tradename MIRANOL, by Miranol, Inc.

Also, suitable amphoteric surfactants are monocarboxylates or dicarboxylates such as cocamphocarboxypropionate, cocoamphocarboxypropionic acid, cocamphocarboxyglycinate, and cocoamphoacetate.

Other types of amphoteric surfactants include aminoalkanoates of the formula
R—NH(CH₂)ₙCOOM
or iminodialkanoates of the formula:
R—N[(CH₂)ₘCOOM]₂
and mixtures thereof, wherein n and m are 1 to 4, R is C₈₋₂₂ alkyl or alkenyl, and M is hydrogen, alkali metal, alkaline earth metal, ammonium or alkanolammonium. Examples of such amphoteric surfactants include n-alkylaminopropionates and n-alkyliminodipropionates, which are sold under the trade name MIRATAINE by Miranol, Inc. or DERIPHAT by Henkel, for example N-lauryl-beta-amino propionic acid, N-lauryl-beta-imino-dipropionic acid, or mixtures thereof.

Zwitterionic surfactants are also suitable for use in the compositions of the invention. The general formula for such surfactants is: 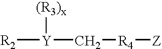
wherein R₂ contains an alkyl, alkenyl or hydroxy alkyl radical of from about 8 to about 18 carbon atoms, from 0 to about 10 ethylene oxide moieties and 0 or I glyceryl moiety; Y is selected from the group consisting of nitrogen, phosphorus, and sulfur atoms; R₃ is an alkyl or monohydroxyalkyl group containing about 1 to 3 carbon atoms; X is 1 when Y is a sulfur atom, and 2 when Y is a nitrogen or phosphorus atom; R₄ is an alkylene or hydroxyalkylene of from about 1 to about 4 carbon atoms, and Z is a radical selected from the group consisting of carboxylate, sulfonate, sulfate, phosphonate, and phosphate groups.

Zwitterionic surfactants include betaines, for example higher alkyl betaines such as coco dimethyl carboxymethyl betaine, lauryl dimethyl carboxymethyl betaine, lauryl dimethyl alphacarboxyethyl betaine, cetyl dimethyl carboxymethyl betaine, lauryl bis-(2-hydroxyethyl)carboxymethyl betaine, stearyl bis-(2-hydroxypropyl)carboxymethyl betaine, oleyl dimethyl gamma-carboxylethyl betaine, and mixtures thereof. Also suitable are sulfo- and amido-betaines such as coco dimethyl sulfopropyl betaine, stearyl dimethyl sulfopropyl betaine, and the like.

C. Sunscreens

1. UVA Chemical Sunscreens

If desired, the composition may comprise one or more UVA sunscreens. The term “UVA sunscreen” means a chemical compound that blocks UV radiation in the wavelength range of about 320 to 400 nm. Preferred UVA sunscreens are dibenzoylmethane compounds having the general formula: 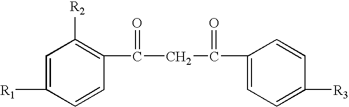
wherein R₁ is H, OR and NRR wherein each R is independently H, C₁₋₂₀ straight or branched chain alkyl; R₂ is H or OH; and R₃ is H, C₁₋₂₀ straight or branched chain alkyl.

Preferred is where R₁ is OR where R is a C ₁₋₂₀ straight or branched alkyl, preferably methyl; R₂ is H; and R₃ is a C₁₋₂₀ straight or branched chain alkyl, more preferably, butyl.

Also suitable are triazine sunscreen agents sold under the trade name Tinosorb®. Examples are Tinosorb® S, having the name ethylhexyloxyphenol methoxyphenyl triazine, and Tinosorb® M, which has the chemical name methylene bis-benzotriaolyl tetramethylbutylphenol.

Examples of particularly suitable UVA sunscreen compounds of the formula above include 4-methyldibenzoylmethane, 2-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′diisopropylbenzoylmethane, 4-tert-butyl-4′-methoxydibenzoylmethane, 4,4′-diisopropylbenzoyl methane, 2-methyl- 5-isopropyl -4′-methoxydibenzoymethane, 2-methyl-5-tert-butyl-4′-methoxydibenzoylmethane, and so on. Particularly preferred is 4-tert-butyl-4′methoxydibenzoylmethane, also referred to as Avobenzone. Avobenzone is commercial available from Givaudan-Roure under the trademark Parsol 1789, and Merck & Co. under the tradename Eusolex 9020.

The composition may contain from about 0.001-20%, preferably 0.005-5%, more preferably about 0.005-3% by weight of the composition of UVA sunscreen. In the preferred embodiment of the invention the UVA sunscreen is Avobenzone, and it is present at not greater than about 3% by weight of the total composition.

2. UVB Chemical Sunscreens

The term “UVB sunscreen” means a compound that blocks UV radiation in the wavelength range of from about 290 to 320 nm. A variety of UVB chemical sunscreens exist including α-cyano-β,β-diphenyl acrylic acid esters as set forth in U.S. Pat. No. 3,215,724, which is hereby incorporated by reference in its entirety. One particular example of α-cyano-β,β-diphenyl acrylic acid ester is Octocrylene, which is 2-ethylhexyl 2-cyano-3,3-diphenylacrylate. In certain cases the composition may contain no more than about 10% by weight of the total composition of octocrylene. Suitable amounts range from about 0.001-10% by weight. Octocrylene may be purchased from BASF under the tradename Uvinul N-539.

Other suitable sunscreens include benzylidene camphor derivatives as set forth in U.S. Pat. No. 3,781,417, which is hereby incorporated by reference in its entirety. Such benzylidene camphor derivatives have the general formula: 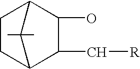
wherein R is p-tolyl or styryl, preferably styryl. Particularly preferred is 4-methylbenzylidene camphor, which is a lipid soluble UVB sunscreen compound sold under the tradename Eusolex 6300 by Merck.

Also suitable are cinnamate derivatives having the general formula; 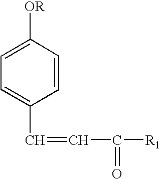
wherein R and R₁ are each independently a C₁₋₂₀ straight or branched chain alkyl. Preferred is where R is methyl and R₁ is a branched chain C₁₋₁₀, preferably C₈ alkyl. The preferred compound is ethylhexyl methoxycinnamate, also referred to as Octoxinate or octyl methoxycinnamate. The compound may be purchased from Givaudan Corporation under the tradename Parsol MCX, or BASF under the tradename Uvinul MC 80. Also suitable are mono-, di-, and triethanolamine derivatives of such methoxy cinnamates including diethanolamine methoxycinnamate. Cinoxate, the aromatic ether derivative of the above compound is also acceptable. If present, the Cinoxate should be found at no more than about 3% by weight of the total composition.

Also suitable as UVB screening agents are various benzophenone derivatives having the general formula: 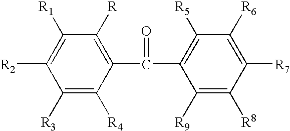
wherein R through R₉ are each independently A, OH, NaO₃S, SO₃H, SO₃Na, Cl, R″, OR″ where R″ is C₁₋₂₀ straight or branched chain alkyl. Examples of such compounds include Benzophenone 1, 2, 3, 4, 5, 6, 7, 8, 9, 10, 11, and 12. Particularly preferred is where the benzophenone derivative is Benzophenone 3 (also referred to as Oxybenzone), Benzophenone 4 (also referred to as Sulisobenzone), Benzophenone 5 (Sulisobenzone Sodium), and the like. Most preferred is Benzophenone 3.

Also suitable are certain menthyl salicylate derivatives having the general formula; 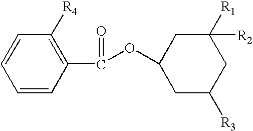
wherein R₁, R₂, R₃, and R₄ are each independently H, OH, NH₂, or C₁₋₂₀ straight or branched chain alkyl. Particularly preferred is where R₁, R₂, and R₃ are methyl and R₄ is hydroxyl or NH₂, the compound having the name homomenthyl salicylate (also known as Homosalate) or menthyl anthranilate. Homosalate is available commercially from Merck under the tradename Eusolex HMS and menthyl anthranilate is commercially available from Haarmann & Reimer under the tradename Heliopan. If present, the Homosalate should be found at no more than about 15% by weight of the total composition.

Various amino benzoic acid derivatives are suitable UVB absorbers including those having the general formula: 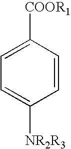
wherein R₁, R₂, and R₃ are each independently H, C₁₋₂₀ straight or branched chain alkyl which may be substituted with one or more hydroxy groups. Particularly preferred is wherein R₁ is H or C₁₋₈ straight or branched alkyl, and R₂ and R₃ are H, or C₁₋₈ straight or branched chain alkyl. Particularly preferred are PABA, ethyl hexyl dimethyl PABA (Padimate O), ethyldihydroxypropyl PABA, and the like. If present Padimate O should be found at no more than about 8% by weight of the total composition.

Salicylate derivatives are also acceptable UVB absorbers. Such compounds have the general formula: 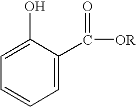
wherein R is a straight or branched chain alkyl, including derivatives of the above compound formed from mono-, di-, or triethanolamines. Particular preferred are octyl salicylate, TEA-salicylate, DEA-salicylate, and mixtures thereof.

Generally, the amount of the UVB chemical sunscreen present may range from about 0.001-45%, preferably 0.005-40%, more preferably about 0.01-35% by weight of the total composition.

3. Physical Sunscreens

The composition may also include one or more physical sunscreens. The term “physical sunscreen” means a material that is generally particulate in form that is able to block UV rays by forming an actual physical block on the skin. Examples of particulates that serve as solid physical sunblocks include titanium dioxide, zinc oxide and the like in particle sizes ranging from about 0.001-150 microns.

If desired, the compositions of the invention may be formulated to have a certain SPF (sun protective factor) values ranging from about 1-50, preferably about 2-45, most preferably about 5-30. Calculation of SPF values is well known in the art. Preferably, the claimed compositions have SPF values greater than 4.

D. Humectants

If desired, the compositions of the invention comprise 0.01-30O%, preferably 0.5-25%, more preferably 1-20% by weight of the total composition of one or more humectants. Suitable humectants include materials such as glycols, sugars, and the like. Suitable glycols include polyethylene and polypropylene glycols such as PEG 4-240, which are polyethylene glycols having from 4 to 240 repeating ethylene oxide units; as well as C₁-₆ alkylene (glycols such as propylene glycol, butylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on, Preferably, the humectants used in the composition of the invention are C₁₋₆, preferably C₂₋₄ alkylene glycols, most particularly butylene glycol.

E. Botanical Extracts

It may be desirable to include one or more botanical extracts in the compositions. If so, suggested ranges are from about 0.0001 to 10%, preferably about 0.0005 to 8%, more preferably about 0.001 to 5% by weight of the total composition, Suitable botanical extracts include extracts from plants (herbs, roots, flowers, fruits, seeds) such as flowers, fruits, vegetables, and so on, including acacia (dealbata, farnesiana, senegal), acer saccharinum (sugar maple), acidopholus, acorus, aesculus, agaricus, agave, agrimonia, algae, aloe, citrus, brassica, cinnamon, orange, apple, blueberry, cranberry, peach, pear, lemon, lime, pea, seaweed, green tea, chamomile, willowbark, mulberry, poppy, and those set forth on pages 1646 through 1660 of the Cosmetic Ingredient Handbook, Eighth Edition, Volume 2. Further specific examples include, but are not limited to, Glycyrrhiza Glabra, Salix Nigra, Macrocycstis Pyrifera, Pyrus Mals, Saxifraga Sarmentosa, Vitis Vinifera, Morus Nigra, Scutellaria Baicalensis, Anthemis Nobilis, Salvia Sclarea, Rosmarinus Officianalis, Citrus Medica Limonum, and mixtures thereof.

F. Structuring Agents

The compositions of the invention may comprise one more structuring agents. The term “structuring agent” means an ingredient or combination of ingredients that increase the viscosity of, or thicken, the composition. Suggested ranges of structuring agent, if present, range from about 0.01-65%, preferably about 0.05-50%, more preferably about 0.1-45% by weight of the total composition. If the composition is in the form of an emulsion, the structuring agent may be found in the oil phase, water phase, or both phases. In the event the composition is anhydrous, the structuring agent may be found in the oil phase of the composition, or as part of the particulate phase, etc.

1. Montmorillonite Minerals

One type of structuring agent that may be used in the composition comprises natural or synthetic montmorillonite minerals such as hectorite, bentonite, and quaternized derivatives thereof, which are obtained by reacting the minerals with a quaternary ammonium compound, such as stearalkonium bentonite, hectorites, quaternized hectorites such as Quaternium-18 hectorite, attapulgite, carbonates such as propylene carbonate, bentones, and the like. Particularly preferred is Quaternium-18 hectorite.

2. Associative Thickeners

Also suitable as structuring agents are various polymeric compounds known in the art as associative thickeners. Suitable associative thickeners generally contain a hydrophilic backbone and hydrophobic side groups. Examples of such thickeners include polyacrylates with hydrophobic side groups, cellulose ethers with hydrophobic side groups, polyurethane thickeners. Examples of hydrophobic side groups are long chain alkyl groups such as dodecyl, hexadecyl, or octadecyl; alkylaryl groups such as octylphenyl or nonyphenyl. Further specific examples include hydroxypropylcellulose, hydroxypropylethylcellulose, cellulose gums, and the like.

3. Silicas and Silicates

Another type of structuring agent that may be used in the compositions are silicas, silicates, silica silylate, and alkali metal or alkaline earth metal derivatives thereof. These silicas and silicates are generally found in the particulate form and include silica, silica silylate, magnesium aluminum silicate, and the like.

4. Silicone Elastomers

Also suitable as structuring agents are cross-linked organosiloxane compounds also known as silicone elastomers. Such elastomers are generally prepared by reacting a dimethyl metlhylhydrogen siloxane with a crosslinking group comprised of a siloxane having an alkylene group having terminal olefinic unsaturation, or with an organic group having an alpha or omega diene. Examples of suitable silicone elastomers for use as thixotropic agents include Dow Corning 9040, sold by Dow Corning, and various elastomeric silicones sold by Shin-Etsu under the KSG tradename including KSG 15, KSG 16, KSG 19 and so on.

5. Natural or Sythetic Organic Waxes

Suitable structuring agents include natural or synthetic waxes. A variety of waxes are suitable including animal, vegetable, mineral, or silicone waxes. Generally such waxes have a melting point ranging from about 28 to 125° C., preferably about 30 to 100° C. Examples of waxes include acacia, beeswax, ceresin, cetyl esters, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, candelilla, grape wax, and polyalkylene glycol derivatives thereof such as PEG6-20 beeswax, or PEG-12 carnauba wax; or fatty acids or fatty alcohols, including esters thereof, such as hydroxystearic acids (for example 12-hydroxy stearic acid), tristearin, tribehenin, and so on.

6. Silicone Waxes

Also suitable are various types of silicone waxes, referred to as alkyl silicones, which are polymers that comprise repeating dimethylsiloxy units in combination with one or more methyl-long chain alkyl siloxy units wherein the long chain alkyl is generally a fatty chain that provides a wax-like characteristic to the silicone such that is a solid or semi-solid at room temperature. Such silicones include, but are not limited to stearoxydimethicone, behenoxy dimethicone, stearyl dimethicone, cetearyl dimethicone, and so on. Suitable waxes are set forth in U.S. Pat. No. 5,725,845, which is hereby incorporated by reference in its entirety.

7. Polyamides and Silicone Polyamides

Also suitable as structuring agents are various types of polyamides or silicone polyamides including those set forth in U.S. patent publication nos. 2002/0114773 or 2003/0072730, both of which are hereby incorporated by reference in their entirety.

Silicone polyamides include those having moieties of the general formula: 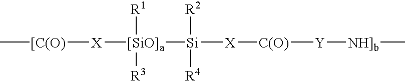
wherein:

X is a linear or branched alkylene having from about 1-30 carbon atoms,

R¹, R², R³ and R⁴ are each independently C₁₋₃₀ straight or branched chain alkyl which may be substituted with one or more hydroxyl or halogen groups; phenyl which may be substituted with one or more C₁₋₃₀ alkyl groups, halogen, hydroxyl, or alkoxy groups; or a siloxane chain having the general formula:

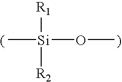

Y is:

(a) a linear or branched alkylene having from about 1-40 carbon atoms which may be substituted with (i) one or more amide groups having the general formula R¹CONR¹, or (ii) C₅₋₆ cyclic ring, or (iii) phenylene which may be substituted with one or more C₁₋₁₀ alkyl groups, or (iv) hydroxy, or (v) C₃₋₈ cycloalkane, or (vi) C₁₋₂₀ alkyl which may be substituted with one or more hydroxy groups, or (vii) C₁₋₁₀ alkyl amines; or

(b) TR⁵R⁶R⁷

wherein R⁵, R⁶, and R⁷, are each independently a C₁₋₁₀ linear or branched alkylene, and T is CR⁸ wherein R⁸ is hydrogen, a trivalent atom N, P, or Al, or a C₁₋₃₀ straight or branched chain alkyl which may be substituted with one or more hydroxyl or halogen groups; phenyl which may be substituted with one or more C₁₋₃₀ alkyl groups, halogen, hydroxyl, or alkoxy groups; or a siloxane chain having the general formula;

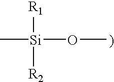

and a and b are each independently sufficient to provide a silicone polyamide polymer having a melting point ranging from about 60 to 120° C., preferably about 85 to 105° C. and a molecular weight ranging from about 40,000 to 500,000 Daltons, preferably about 65,000 to 149,000 Daltons.

Preferred is where R¹, R², R³, and R⁴ are C₁₋₁₀, preferably methyl; and X and Y is a linear or branched alkylene. Preferred are silicone polyamides having the general formula: 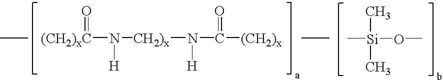
wherein a, b, and x are each independently sufficient to provide a silicone polyamide polymer having a melting point ranging from about 60 to 120° C., preferably about 85 to 105° C. and a molecular weight ranging from about 40,000 to 500,000 Daltons, preferably about 65,000 to 149,000 Daltons One type of silicone polyamide that may be used in the compositions of the invention may be purchased from Dow Corning Corporation under the tradename Dow Corning 2-8178 gellant which has the INCI name nylon-611/dimethicone copolymer which is sold in a composition containing PPG-3 myristyl ether.

G. Particulate Materials

The compositions of the invention may contain particulate materials in the form of pigments, inert particulates, or mixtures thereof. If present, suggested ranges are from about 0.01-75%, preferably about 0.05-70%, more preferably about 0.1-65% by weight of the total composition. In the case where the composition may comprise mixtures of pigments and powders, suitable ranges include about 0.01-75% pigment and 0.1-75% powder, such weights by weight of the total composition.

1. Powders

The particulate matter may be colored or non-colored (for example white) non-pigmentatious powders. Suitable non-pigmentatious powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof. The above mentioned powders may be surface treated with lecithin, amino acids, mineral oil, silicone, or various other agents either alone or in combination, which coat the powder surface and render the particles more lipophilic in nature.

2. Pigments

The particulate materials may comprise various organic and/or inorganic pigments. The organic pigments are generally various aromatic types including azo, indigoid, triphenylmethane, anthroquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof. Iron oxides of red, blue, yellow, brown, black, and mixtures thereof are suitable

H. Film Forming Polymers

The compositions of the invention may comprise one or more film forming polymers that aid in forming a film on the skin or provide other effects that lend beneficial properties to the formula. Examples of such film forming polymers include, but are not limited to those set forth below.

Silicone Film Forming Polymers

(a) Siloxane Polymeric Resins and Gums

Siloxane polymeric resins that comprises tetrafunctional or trifunctional units either alone or in combination with monofunctional units are suitable silicone film forming polymers for use in the composition. The term “siloxane polymeric resin” means that the siloxane is a polymer, or is comprised of repeating units or “mers”.

The term “resin” means that the siloxane polymer provides substantive, resinous, film forming properties when applied to skin. In the context of this invention, the term “resin” will mean a siloxane containing enough cross-linking to provide substantive, film forming properties. The term cross-linking means a moiety where the silicon atom is bonded to at least three, preferably four oxygen atoms when the moiety is polymerized with another siloxane unit.

The term “film forming” means that the siloxane resin is capable of forming a film, in particular, a substantive film, on the keratinous surface to which it is applied.

The term monofunctional unit means a siloxy unit that contains one silicon atom bonded to one oxygen atom, with the remaining three substituents on the silicon atom being other than oxygen. In particular, in a monofunctional siloxy unit, the oxygen atom present is shared by 2 silicon atoms when the monofunctional unit is polymerized with one or more of the other units. In silicone nomenclature used by those skilled in the art, a monofunctional siloxy unit is designated by the letter “M”, and means a unit having the general formula:
R₁R₂R₃SiO_{1/2}
wherein R₁, R₂, and R₃ are each independently C₁₋₃₀ preferably C₁₋₁₀ , more preferably C₁₋₄ straight or branched chain alkyl, which may be substituted with phenyl or one or more hydroxyl groups; phenyl; alkoxy (preferably C1-22, more preferably C₁₋₆); or hydrogen. The SiO_{1/2} designation means that the oxygen atom in the monofunctional unit is bonded to, or shared, with another silicon atom when the monofunctional unit is polymerized with one or more of the other types of units. For example, when R₁, R₂, and R₃ are methyl the resulting monofunctional unit is of the formula:

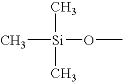

When this monofunctional unit is polymerized with one or more of the other units the oxygen atom will be shared by another silicon atom, i.e. the silicon atom in the monofunctional unit is bonded to ½ of this oxygen atom.

The term “difunctional siloxy unit” is generally designated by the letter “D” in standard silicone nomenclature. If the D unit is substituted with substituents other than methyl the “D” designation is sometimes used, which indicates a substituent other than methyl. For purposes of this disclosure, a “D” unit has the general formula:
R₁R₂SiO_{2/2}
wherein R₁ and R₂ are defined as above. The SiO_{2/2} designation means that the silicon atom in the difunctional unit is bonded to two oxygen atoms when the unit is polymerized with one or more of the other units. For example, when R₁ and R₂, are methyl the resulting difunctional unit is of the formula: 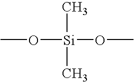
When this difunctional unit is polymerized with one or more of the other units the silicon atom will be bonded to two oxygen atoms, i.e. will share two one-halves of an oxygen atom.

The tern “trifunctional siloxy unit” is generally designated by the letter “T” in standard silicone nomenclature. A “T” unit has the general formula;
R₁SiO_{3/2}
wherein R₁ is as defined above. The SiO_{3/2} designation means that the silicon atom is bonded to three oxygen atoms when the unit is copolymerized with one or more of the other units. For example when R₁ is methyl the resulting trifunctional unit is of the formula: 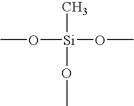
When this trifunctional unit is polymerized with one or more of the other units, the silicon atom shares three oxygen atoms with other silicon atoms, i.e. will share three halves of an oxygen atom.

The term “tetrafunctional siloxy unit” is generally designated by the letter “Q” in standard silicone nomenclature. A “Q” unit has the general formula:
SiO_{4/2}

The SiO_{4/2} designation means that the silicon shares four oxygen atoms (i.e., four halves) with other silicon atoms when the tetrafunctional unit is polymerized with one or more of the other units. The SiO_{4/2} unit is best depicted as follows:

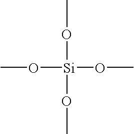

The film forming siloxane resins that may be used in the compositions of the invention comprises D, T or Q units either alone or in combination with M units. In addition, there may be one or more of the other types of units present in the polymer.

The film forming polymeric siloxane resin may be a liquid, semi-solid, or solid at room temperature. Preferably, the siloxane polymeric resin is a semi-solid or solid at room temperature.

Typically T or MT silicones are referred to as silsesquioxanes, and in the case where M units are present methylsilsesquioxanes. Preferred are T silicones having the following general formula:
(R₁SiO_{3/2})X
where x ranges from about 1 to 100,000, preferably about 1-50,000, more preferably about 1-10,000, and wherein R₁ is as defined above. Such MT silicones are generally referred to as polymethylsilsesquioxane which are silsesquioxanes containing methyl groups.

Examples of specific polysilsesquioxanes that may be used are manufactured by Wacker Chemie under the Resin MK designation. This polysilsesquioxane is a polymer comprised of T units and, optionally one or more D (preferably dimethylsiloxy) units. This particularly polymer may have ends capped with ethoxy groups, and/or hydroxyl groups, which may be due to how the polymers are made. e.g. condensation in aqueous or alcoholic media. Other suitable polysilsesquioxanes that may be used as the film forming polymer include those manufactured by Shin-Etsu Silicones and include the “KR” series, e.g. KR-220L, 242A, and so on. These particular silicone resins may contain endcap units that are hydroxyl or alkoxy groups which may be present due to the manner in which such resins are manufactured.

Also suitable are MQ resins, which are siloxy silicate polymers having the following general formula: 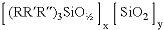
wherein R, R′and R″ are each independently a C₁₋₁₀ straight or branched chain alkyl or phenyl, and x and y are such that the ratio of (RR′R″)₃SiO_{1/2} units to SiO₂ units ranges from about 0.5 to 1 to 1.5 to 1. Preferably R, R′ and R″ are a C₁₋₆ alkyl, and more preferably are methyl and x and y are such that the ratio of (CH₃)₃SiO_{1/2} units to SiO₂ units is about 0.75 to 1. Most preferred is this trimethylsiloxysilicate containing 2.4 to 2.9 weight percent hydroxyl groups which is formed by the reaction of the sodium salt of silicic acid, chlorotrimethylsilane, and isopropyl alcohol. The manufacture of trimethylsiloxysilicate is set forth in U.S. Pat. Nos. 2,676,182; 3,541,205; and 3,836,437, all of which are hereby incorporated by reference. Trimethylsiloxysilicate as described is available from GE Silicones under the tradename SR-1000 which is a solid particulate material. Also suitable is Dow Corning 749 which is a mixture of volatile cyclic silicone and trimethylsiloxysilicate.

The film forming siloxane polymeric resins that may be used in the composition are made according to processes well known in the art. In general siloxane polymers are obtained by hydrolysis of silane monomers, preferably chlorosilanes. The chlorosilanes are hydrolyzed to silanols and then condensed to form siloxanes. For example, Q units are often made by hydrolyzing tetrachlorosilanes in aqueous or aqueous/alcoholic media to form the following: 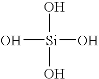
The above hydroxy substituted silane is then condensed or polymerized with other types of silanol substituted units such as: 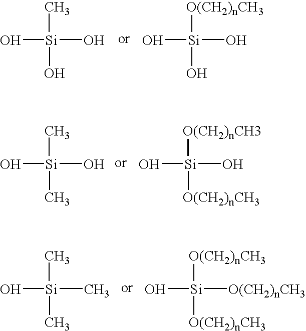
wherein n is 0-10, preferably 0-4.

Because the hydrolysis and condensation may take place in aqueous or aqueous/alcoholic media wherein the alcohols are preferably lower alkanols such as ethanol, propanol, or isopropanol, the units may have residual hydroxyl or alkoxy functionality as depicted above. Preferably, the resins are made by hydrolysis and condensation in aqueous/alcoholic media, which provides resins that have residual silanol and alkoxy functionality. In the case where the alcohol is ethanol, the result is a resin that has residual hydroxy or ethoxy functionality on the siloxane polymer. The silicone film forming polymers used in the compositions of the invention are generally made in accordance with the methods set forth in Silicon Compounds (Silicones), Bruce B. Hardman, Arnold Torkelson, General Electric Company, Kirk-Othmer Encyclopedia of Chemical Technology, Volume 20, Third Edition, pages 922-962, 1982, which is hereby incorporated by reference in its entirety.

Also suitable are linear, high molecular weight silicones that are semi-solids, solids, or gums at room temperature. Examples of such silicones include dimethicones having viscosities ranging from about 100,000 to 10 million, or 500,000 to 10 million centipoise or dimethicone copolyols having the same viscosity range.

Also suitable are silicone esters as disclosed in U.S. Pat. Nos. 4,725,658 and 5,334,737, which are hereby incorporated by reference. Such silicone esters comprise units of the general formula RₐR^{E}_{b}SiO_{[4-(a+b)/2]} or R¹³ₓR^{E}_{y}SiO_{1/2}, wherein R and R¹³ are each independently an organic radical such as alkyl, cycloalkyl, or aryl, or, for example, methyl, ethyl, propyl, hexyl, octyl, decyl, aryl, cyclohexyl, and the like, a is a number ranging from 0 to 3, b is a number ranging from 0 to 3, a+b is a number ranging from 1 to 3, x is a number from 0 to 3, y is a number from 0 to 3 and the sum of x+y is 3, and wherein R^{E} is a carboxylic ester containing radical. Preferred R^{E} radicals are those wherein the ester group is formed of one or more fatty acid moieties (e.g. of about 2, often about 3 to 10 carbon atoms) and one or more aliphatic alcohol moieties (e.g. of about 10 to 30 carbon atoms). Examples of such acid moieties include those derived from branched-chain fatty acids such as isostearic, or straight chain fatty acids such as behenic. Examples of suitable alcohol moieties include those derived from monohydric or polyhydric alcohols, e.g. normal alkanols such as n-propanol and branched-chain etheralkanols such as (3,3,3-trimethylolpropoxy)propane. Preferably the ester subgroup (i.e. the carbonyloxy radical) will be linked to the silicon atom by a divalent aliphatic chain that is at least 2 or 3 carbon atoms in length, e.g. an alkylene group or a divalent alkyl ether group. Most preferably that chain will be part of the alcohol moiety, not the acid moiety. Such silicones may be liquids or solids at room temperature.

(b). Copolymers of Silicone and Ethylenically Unsaturated Monomers

Another type of film forming polymer that may be used in the compositions of the invention is obtained by reacting silicone moieties with ethylenically unsaturated monomers. The resulting copolymers may be graft or block copolymers. The term “graft copolymer” is familiar to one of ordinary skill in polymer science and is used herein to describe the copolymers which result by adding or “grafting” polymeric side chain moieties (i.e. “grafts”) onto another polymeric moiety referred to as the “backbone”. The backbone may have a higher molecular weight than the grafts. Thus, graft copolymers can be described as polymers having pendant polymeric side chains, and which are formed from the “grafting” or incorporation of polymeric side chains onto or into a polymer backbone. The polymer backbone can be a homopolymer or a copolymer. The graft copolymers are derived from a variety of monomer units.

One type of polymer that may be used as the film forming polymer is a vinyl-silicone graft or block copolymer having the formula: 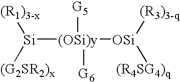
wherein G₅ represents monovalent moieties which can independently be the same or different selected from the group consisting of alkyl, aryl, aralkyl, alkoxy, alkylamino, fluoroalkyl, hydrogen, and -ZSA;

wherein A represents a vinyl polymeric segment consisting essentially of a polymerized free radically polymerizable monomer, and Z is a divalent linking group such as C₁₋₁₀ alkylene, aralkylene, arylene, and alkoxylalkylene, most preferably Z is methylene or propylene,

G₆ is a monovalent moiety which can independently be the same or different selected from the group consisting of alkyl, aryl, aralkyl, alkoxy, alkylamino, fluoroalkyl, hydrogen, and -ZSA;

G₂ comprises A;

G₄ comprises A;

R₁ is a monovalent moiety which can independently be the same or different and is selected from the group consisting of alkyl aryl, aralkyl, alkoxy, alkylamino, fluoroalkyl, hydrogen, and hydroxyl; but preferably C₁₋₄ alkyl or hydroxyl, and most preferably methyl.

R₂ is independently the same or different and is a divalent linking group such as C₁₋₁₀ alkylene, arylene, aralkylene, and alkoxyalkylene, preferably C₁₋₃ alkylene or C₇₋₁₀ aralkylene, and most preferably —CH₂— or 1,3-propylene,

R₃ is a monovalent moiety which is independently alkyl, aryl, aralkyl, alkoxy, alkylamino, fluoroalkyl, hydrogen, or hydroxyl, preferably C₁₋₄ alkyl or hydroxyl, most preferably methyl;

R₄ is independently the same or different and is a divalent linking group such as C₁₋₁₀ alkylene, arylene, aralkylene, alkoxyalkylene, but preferably C₁₋₃ alkylene and C₇₋₁₀ alkarylene, most preferably —CH₂— or 1,3-propylene,

x is an integer of 0-3;

y is an integer of 5 or greater; preferably 10 to 270, and more preferably 40-270; and

q is an integer of 0-3.

These polymers are described in U.S. Pat. No. 5,468,477, which is hereby incorporated by reference. Most preferred is poly(dimethylsiloxane)-g-poly(isobutyl methacrylate), which is manufactured by 3-M Company under the tradename VS 70 IBM. This polymer may be purchased in the dry particulate form, or as a solution where the polymer is dissolved in one or more solvents such as isododecane. Preferred is where the polymer is in dry particulate form, and as such it can be dissolved in one or more of the liquids comprising the liquid carrier. This polymer has the CTFA name Polysilicone-6.

Another type of such a polymer comprises a vinyl, methacrylic, or acrylic backbone with pendant siloxane groups and pendant fluorochemical groups. Such polymers preferably comprise repeating A, C, D and optionally B monomers wherein:

A is at least one free radically polymerizable acrylic or methacrylic ester of a 1,1,-dihydroperfluoroalkanol or analog thereof, omega-hydridofluoroalkanols, fluoroalkylsulfonamido alcohols, cyclic fuoroalkyl alcohols, and fluoroether alcohols,

B is at least one reinforcing monomer copolymerizable with A,

C is a monomer having the general formula X(Y)nSi(R)3-mZₘ wherein

X is a vinyl group copolymerizable with the A and B monomers,

Y is a divalent linking group which is alkylene, arylene, alkarylene, and aralkylene of 1 to 30 carbon atoms which may incorporate ester, amide, urethane, or urea groups,

n is zero or 1;

m is an integer of from 1 to 3,

R is hydrogen, C₁₋₄ alkyl, aryl, or alkoxy,

Z is a monovalent siloxane polymeric moiety; and

D is at least one free radically polymerizable acrylate or methacrylate copolymer.

Such polymers and their manufacture are disclosed in U.S. Pat. Nos. 5,209,924 and 4,972,037, which are hereby incorporated by reference. More specifically, the preferred polymer is a combination of A, C, and D monomers wherein A is a polymerizable acrylic or methacrylic ester of a fluoroalkylsulfonamido alcohol, and where D is a methacrylic acid ester of a C_{1/2} straight or branched chain alcohol, and C is as defined above. Most preferred is a polymer having moieties of the general formula: 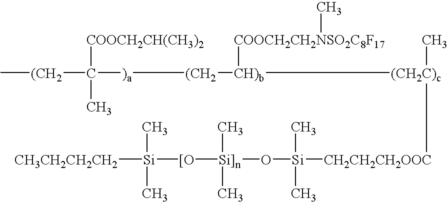
wherein each of a, b, and c has a value in the range of 1-100,000, n has a value preferably in the range of 1-1,000,000, and the terminal groups are selected from the group consisting of a C₁₋₂₀ straight or branched chain alkyl, aryl, ad alkoxy and the like. These polymers may be purchased from Minnesota Mining and Manufacturing Company under the tradenames “Silicone Plus” polymers. Most preferred is poly(isobutyl methacrylate-co-methyl FOSEA)-g-poly(dimethylsiloxane) which is sold under the tradename SA 70-5 IBMMF.

Another suitable silicone acrylate copolymer is a polymer having a vinyl, methacrylic, or acrylic polymeric backbone with pendant siloxane groups. Such polymers as disclosed in U.S. Pat. Nos. 4,693,935, 4,981,903, 4,981,902, and which are hereby incorporated by reference. Preferably, these polymers are comprised of A, C, and optionally B monomers wherein:

A is at least one free radically polymerizable vinyl, methacrylate, or acrylate monomer;

B, when present, is at least one reinforcing monomer copolymerizable with A,

C is a monomer having the general formula:
X(Y)ₙSi(R)₃₋ₘZₘ
wherein:

X is a vinyl group copolymerizable with the A and B monomers;

Y is a divalent linking group;

n is zero or 1;

m is an integer of from 1 to 3;

R is hydrogen, C₁₋₁₀ alkyl, substituted or unsubstituted phenyl, C₁₋₁₀ alkoxy; and

Z is a monovalent siloxane polymeric moiety.

Examples of A monomers are lower to intermediate methacrylic acid esters of C₁₋₁₂ straight or branched chain alcohols, styrene, vinyl esters, vinyl chloride, vinylidene chloride, acryloyl monomers, and so on.

The B monomer, if present, is a polar acrylic or methacrylic monomer having at least one hydroxyl, amino, or ionic group (such as quaternary ammonium, carboxylate salt, sulfonic acid salt, and so on).

The C monomer is as above defined.

Examples of other suitable copolymers that may be used herein, and their method of manufacture, are described in detail in U.S. Pat. No. 4,693,935, Mazurek and U.S. Pat. No. 4,728,571, Clemens et al., both of which are incorporated herein by reference. Additional grafted polymers are also disclosed in EPO application 90307528.1, published as EPO application 0 408 311, U.S. Pat. No. 5,061,481, Suzuki et al., U.S. Pat. No. 5,106,609, Bolich et al. U.S. Pat. No. 5,100,658, Bolich et al., U.S. Pat. No. 5,100,657, Ansher-Jackson et al., U.S. Pat. No. 5,104,646, Bolich et al., U.S. Pat. No. 5,618,524, issued Apr. 8, 1997, all of which are incorporated by reference herein in their entirety.

(c). Synthetic Organic Polymers

Also suitable for use as film forming polymers in the compositions are polymers made by polymerizing one or more ethylenically unsaturated monomers. The final polymer may be a homopolymer, copolymer, terpolymer, or graft or block copolymer, and may contain monomeric units such as acrylic acid, methacrylic acid or their simple esters, styrene, ethylenically unsaturated monomer units such as ethylene, propylene, butylene, etc., vinyl monomers such as vinyl chloride, styrene, and so on.

In some cases, polymers containing one or more monomers which are esters of acrylic acid or methacrylic acid, including aliphatic esters of methacrylic acid like those obtained with the esterification of methacrylic acid or acrylic acid with an aliphatic alcohol of 1 to 30, preferably 2 to 20, more preferably 2 to 8 carbon atoms. If desired, the aliphatic alcohol may have one or more hydroxy groups are particularly suitable. Also suitable are methacrylic acid or acrylic acid esters esterified with moieties containing alicyclic or bicyclic rings such as cyclohexyl or isobornyl, for example.

The ethylenically unsaturated monomer may be mono-, di-, tri-, or polyfunctional as regards the addition-polymerizable ethylenic bonds. A variety of ethylenically unsaturated monomers are suitable.

Examples of suitable monofunctional ethylenically unsaturated monomers include those of the formula. 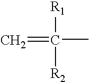
wherein R₁ is H, a C₁₋₃₀ straight or branched chain alkyl, aryl, or aralkyl; R₂ is a pyrrolidone, a C₁₋₃₀ straight or branched chain alkyl, or a substituted or unsubstituted aromatic, alicyclic, or bicyclic ring where the substituents are C₁₋₃₀ straight or branched chain alkyl, or COOM or OCOM wherein M is H, a C₁₋₃₀ straight or branched chain alkyl, pyrrolidone, or a substituted or unsubstituted aromatic, alicyclic, or bicyclic ring where the substituents are C₁₋₃₀ straight or branched chain alkyl which may be substituted with one or more hydroxyl groups, or [(CH₂)ₘO]ₙH wherein m is 1-20, and n is 1-200.

More specific examples include the monofunctional ethylenically unsaturated monomer is of Formula I, above, wherein R₁ is H or a C₁₋₃₀ alkyl, and R₂ is COOM or OCOM wherein M is a C₁₋₃₀ straight or branched chain alkyl which may be substituted with one or more hydroxy groups.

Further examples include where R₁ is H or CH₃, and R₂ is COOM wherein M is a C₁₋₁₀ straight or branched chain alkyl which may be substituted with one or more hydroxy groups.

Di-, tri- and polyfunctional monomers, as well as oligomers, of the above monofunctional monomers may also be used to form the polymer. Suitable difunctional monomers include those having the general formula: 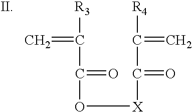
wherein R₃ and R₄ are each independently H, a C₁₋₃₀ straight or branched chain alkyl, aryl, or aralkyl; and X is [(CH₂)ₓO_{y}]_{z} wherein x is 1-20, and y is 1-20, and z is 1-100. Particularly preferred are difunctional acrylates and methacrylates, such as the compound of Formula II above wherein R₃ and R₄ are CH₃ and X is [(CH₂)ₓO_{y}]_{z} wherein x is 1-4; and y is 1-6; and z is 1-10.

Trifunctional and polyfunctional monomers are also suitable for use in the polymerizable monomer to form the polymer used in the compositions of the invention. Examples of such monomers include acrylates and methacrylates such as trimethylolpropane trimethacrylate or trimethylolpropane triacrylate.

The polymers can be prepared by conventional free radical polymerization techniques in which the monomer, solvent, and polymerization initiator are charged over a 1-24 hour period of time, preferably 2-8 hours, into a conventional polymerization reactor in which the constituents are heated to about 60-175° C., preferably 80-100° C. The polymers may also be made by emulsion polymerization or suspension polymerization using conventional techniques. Also anionic polymerization or Group Transfer Polymerization (GTP) is another method by which the copolymers used in the invention may be made. GTP is well known in the art and disclosed in U.S. Pat. Nos. 4,414,372; 4,417,034; 4,508,880; 4,524,196; 4,581,428; 4,588,795; 4,598,161; 4,605,716; 4,605,716; 4,622:372; 4,656,233; 4,711 942; 4,681,918; and 4,822,859; all of which are hereby incorporated by reference.

Also suitable are polymers formed from the monomer of Formula I, above, which are cyclized, in particular, cycloalkylacrylate polymers or copolymers having the following general formulas: 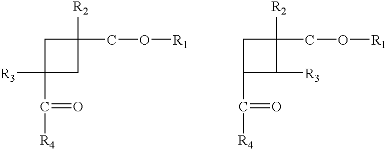
wherein R₁, R₂, R₃, and R₄ are as defined above. Typically such polymers are referred to as cycloalkylacrylate polymers. Such polymers are sold by Phoenix Chemical, Inc. under the tradename Giovarez AC-5099M. Giovarez has the chemical name isododecane acrylates copolymer and the polymer is solubilized in isododecane. The monomers mentioned herein can be polymerized with various types of organic groups such as propylene glycol, isocyanates, amides, etc.

One type of organic group that can be polymerized with the above monomers includes a urethane monomer. Urethanes are generally formed by the reaction of polyhydroxy compounds with diisocyanates, as follows: 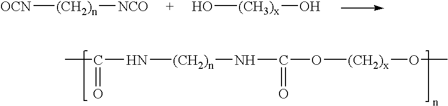
wherein x is 1-1000.

Another type of monomer that may be polymerized with the above comprise amide groups, preferably having the the following formula: 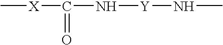
wherein X and Y are each independently linear or branched alkylene having ₁₋₄₀ carbon atoms, which may be substituted with one or more amide, hydrogen, alkyl, aryl, or halogen substituents.

Another type of organic monomer may be alpha or beta pinenes, or terpenes, abietic acid, and the like.

One additional type of synthetic organic polymer that may be used in the compositions of the invention is obtained by polymerizing ethylenically unsaturated monomers which comprise vinyl ester groups either alone or in combination with other monomers including silicone monomers, other ethylenically unsaturated monomers, or organic groups such as amides, urethanes, glycols, and the like. The various types of monomers or moieties may be incorporated into the film forming polymer by way of free radical polymerization, addition polymerization, or by formation of grafts and blocks which are attached to the growing polymer chain according to processes known in the art.

Typically, this type of film forming polymer comprises vinyl ester monomers having the following general formula: 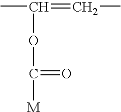
wherein M is H, or a straight or branched chain C₁₋₁₀₀ alkyl, preferably a C₁₋₅₀ alkyl, more preferably a C₁₋₄₅ alkyl which may be saturated or unsaturated, or substituted or unsubstituted, where the substituents include hydroxyl, ethoxy, amide or amine, halogen, alkyloxy, alkyloxycarbonyl, and the like. Preferably, M is H or a straight or branched chain alkyl having from 1 to 30 carbon atoms. The film forming polymer may be a homopolymer or copolymer having the vinyl ester monomers either alone or in combination with other ethylenically unsaturated monomers, organic groups, or silicone monomers.

Suitable other monomers that may be copolymerized with the vinyl ester monomer include those having siloxane groups, including but not limited to repeat units having the formula: 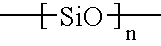
wherein R and R′ are each independently a C1-30 straight or branched chain alkyl, phenyl, or trimethylsiloxy and n ranges from 1-1,000,000. The silicone monomers are preferably polymerized into a siloxane polymer then attached to the polymer chain by attaching a terminal organic group having olefinic unsaturation such as ethylene or propylene, to the siloxane, then reacting the unsaturated group with a suitable reactive site on the polymer to graft the siloxane chain to the polymer.

Also suitable are various types of organic groups that may be polymerized with the vinyl ester monomers including but not limited to urethane, amide, polyalkylene glycols, and the like as set forth above.

The vinyl ester monomers may also be copolymerized with other ethylenically unsaturated monomers that are not vinyl esters, including those set forth above.

(d). Natural Polymers

Also suitable for use are one or more naturally occurring polymeric materials such as resinous plant extracts including such as rosin, shellac, chitin, and the like.

1. Preservatives

The composition may contain 0.001-8%, preferably 0.01-6%, more preferably 0.05-5% by weight of the total composition of preservatives. A variety of preservatives are suitable, including such as benzoic acid, benzyl alcohol, benzylhemiformal, benzylparaben, 5-bromo-5-nitro-1,3-dioxane, 2-bromo-2-nitropropane-1,3-diol, butyl paraben, phenoxyethanol, methyl paraben, propyl paraben, diazolidinyl urea, calcium benzoate, calcium propionate, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, and all of those disclosed on pages 570 to 571 of the CTFA Cosmetic Ingredient Handbook, Second Edition, 1992, which is hereby incorporated by reference.

J. Vitamins and Antioxidants

The compositions of the invention may contain vitamins and/or coenzymes, as well as antioxidants. If so, 0.001-10%, preferably 0.01-8%, more preferably 0.05-5% by weight of the total composition are suggested. Suitable vitamins include ascorbic acid and derivatives thereof, the B vitamins such as thiamine, riboflavin, pyridoxin, and so on, as well as coenzymes such as thiamine pyrophosphate, flavin adenin dinucleotide, folic acid, pyridoxal phosphate, tetrahydrofolic acid and so on. Also Vitamin A and derivatives thereof are suitable. Examples are Vitamin A palmitate, acetate, or other esters thereof, as well as Vitamin A in the form of beta carotene. Also suitable is Vitamin E and derivatives thereof such as Vitamin E acetate, nicotinate, or other esters thereof In addition, Vitamins D and K are suitable.

Suitable antioxidants are ingredients which assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.

III. The Compositions

The cosmetically acceptable carrier for the hexapeptide having Sequence ID No. 1 may be a wide variety of cosmetic compositions including but not limited to creams, lotions, gels, and colored cosmetic compositions such as foundation, lipstick, eyeshadow, blush, concealer, eyeliner, mascara, nail enamel, and the like. Typical ranges of ingredients found in such compositions include, but are not limited to, those set forth herein.

Creams and lotions generally comprise from about 0.1-99% water, 0.1-99% oil, about 0.001-20% of one or more surfactants, and may optionally include any one or more of the ingredients set forth in Section II above. Creams have a more viscous consistency while lotions tend to be less viscous, or more pourable.

Typical foundation makeup compositions and concealers may be found in the emulsion form and will generally comprise from about 0.1-99% water, 0.1-99% oil, about 0.001-20% of one or more surfactants, and from about 0.01-30% of particulate material which may be pigments, powders, or mixtures thereof. The foundation makeup composition may optionally comprise any of the other ingredients described in Section II above, and in the ranges set forth.

Foundation makeup, powder, and concealer compositions may also be in the anhydrous form. If so, typical ranges of ingredients include from about 0.1-75% oil and about 0.1-75% particulate materials, which may be pigments, powders, or mixtures thereof. Such compositions may optionally contain one or more of the ingredients set forth in Section II and in the ranges set forth.

Blushes and eyeshadows may be in the water and emulsion form, and if so, typically contain the ranges of ingredients set forth above with respect to foundation makeup and, optionally, any one or more of the other ingredients set forth in Section II, and in the same amounts. However, blushes and eyeshadows may also be in the anhydrous form and, if so, contain the ranges of ingredients set forth with respect to the anhydrous foundation and powder compositions mentioned above and the optional ingredients listed in Section II, above.

Typically, lipsticks contain from about 0.01-99% oil, 0.1-50% structuring agent, and from about 0.1-50% of particulates which may be pigments, powders, or mixtures thereof. The lipsticks may contain one or more of the ingredients mentioned in Section II and in the same ranges as set forth therein.

Mascara compositions may be in the emulsion form, and if so, typically contain from about 0.1-99% water and from about 0.1-99% oil, and 0.1-50% particulate matter. Optionally, mascaras may contain from about 0.1-50% surfactants, and the other ingredients set forth in Section II above. Mascaras may also be anhydrous, and if so, may comprise from about 0.1-99% oil, 0.1-50% particulate matter, and, optionally, one or more of the ingredients set forth in Section II and in the ranges set forth.

In general, the hexapeptide having Sequence ID No. 1 may be incorporated into any type of cosmetic composition.

The invention will be further described in connection with the following examples which are set forth for the purposes of illustration only.

### EXAMPLE 1

An oil-in-water emulsion facial and body cream with SPF was prepared as follows:

| INGREDIENT | w/w % |
|---|---|
| Butylene glycol | 5.0 |
| Preservatives | 1.73 |
| Magnesium Ascorbyl Phosphate | 0.01 |
| Silica | 0.75 |
| Glycerin | 5.0 |
| Talc | 0.75 |
| Carbomer (2.5% aqueous solution) | 20.0 |
| Octyl methoxycinnamate | 7.5 |
| Octyl salicylate | 3.0 |
| Homosalate | 5.0 |
| Benzophenone-3 (Oxybenzone) | 2.0 |
| 4-tert-butyl methoxydibenzoylmethane (Avobenzone) | 2.0 |
| Dimethicone | 2.0 |
| Cetyl Alcohol | 1.5 |
| Stearyl Alcohol | 0.75 |
| Talc | 0.75 |
| PPG-2 Myristyl Ether Propionate | 4.5 |
| C12-15 Alkyl Benzoate | 1.0 |
| Tocopheryl Acetate | 0.1 |
| Aloe Barbadensis Leaf Extract | 0.1 |
| Retinyl Palmitate | 0.01 |
| Lauryl Lactate | 1.5 |
| Butylene Glycol Dicaprylate/Dicaprate | 5.0 |
| Peg 100 Stearate | 0.75 |
| Polysorbate 60 | 2.6 |
| Sorbitan stearate | 0.9 |
| Triethanolamine | 1.0 |
| Mica, Titanium | 1.0 |
| Glycyrrhia Glabra extract in cyclomethicone | 1.0 |
| Salix Nigra (willowbark) Extract | 1.0 |
| Oleyl alcohol, Dioscorea Villosa (Yam) Root Extract, Glycine | 1.0 |
| Sojo (soybean) sterols | |
| Trifolium Pratense (Clover) Flower Extract, glycerin, butylene | 1.0 |
| glycol, lecithin | |
| Water, glycerin, Macrocystis Pyrifera Extract, hydrolyzed wheat | 1.0 |
| protein | |
| PEG-40 hydrogenated castor oil, Pyrus Malus (apple) | 0.5 |
| Fruit extract | |
| Saxifraga Sarmentosa Extract, Vitis Vinifera (grape) | 0.5 |
| Fruit Extract, butylene glycol, Morus Bombycis (Mulberry) | |
| Root extract, Scutellaria Baicalensis Root extract, | |
| disodium EDTA, water | |
| Methoxypropylgluconamide | 0.3 |
| Sodium hydroxide | 0.050 |
| Hexapeptide-10 | 1.00 |
| Anthemis Nobilis Flower Extract (chamomile Roman), Salvia | 0.3 |
| Sclarea (clary) extract, citrus medica limonum (lemon) peel | |
| extract | |
| Water | QS |

The composition was prepared by heating water, glycols, preservatives, magnesium ascorbyl phosphate, silica, glycerin and tale to 80° C. with sweep mixing. Once uniform, the carbomer solution was added with sweep agitation maintaining a temperature of 80° C. In a separate vessel oil phase ingredients (Octyl methoxycinnamate to Sorbitan stearate) were mixed with propeller agitation and heated to 80° C. Once both phases reached 80° C. the oil phase was transferred into the water phase with fast agitation. Once the transfer was completed the composition was neutralized with triethanolamine, followed by addition of the mica and titanium dioxide. The mixture was homogenized for 15 minutes, then cooled to 50° C. with continuous agitation. Once the bulk was cooled to 50° C. the remaining ingredients were added with mixing. The sodium hydroxide and the hexapeptide were premixed with water (1%) in a container before adding to the mixture. The mixture was then cooled to 30° C. and poured into suitable containers.

### EXAMPLE 2

An oil-in-water emulsion face and body moisturizing cream was prepared as follows:

| INGREDIENT | w/w % |
|---|---|
| Preservatives | 0.65 |
| Beeswax | 1.25 |
| Hydrogenated polyisobutene | 5.00 |
| Sorbitan stearate | 3.00 |
| Hydrogenated coco-glycerides | 1.00 |
| Octyldodecanol | 2.00 |
| Cetearyl ethylhexanoate | 3.00 |
| Capric caprylic triglycerides | 3.00 |
| Glyceryl stearate | 2.00 |
| Cetearyl alcohol, ceteareth-20 | 3.00 |
| Stearic acid | 3.15 |
| Tetradibutyl Pentaeryrityl hydroxyhydrocinnamate | 0.05 |
| Glycerrhiza Glabra (licorice) Extract in | 1.00 |
| cyclomethicone | |
| Cyclomethicone | 1.00 |
| Triethanolamine | 0.79 |
| Phenoxyethanol | 1.00 |
| Oleyl alcohol, Dioscorea Villosa (wild yam) | 1.00 |
| Root Extract, Glycine Soja (soybean) sterols | |
| Hexapeptide-10 | 1.00 |
| Water | QS |

The cream was prepared by heating the water, preservatives, and magnesium ascorbyl phosphate, glycerin and glycols to 80° C. with sweep mixing. Once uniform, the carbomer solution was added with sweep agitation maintaining the temperature at 80° C. In a separate vessel the oil phase ingredients (dimethicone through glyceryl stearate, PEG 100 stearate in the above formula) were mixed with propeller agitation and heated to 80° C. Once both phases reached 80° C. the oil phase was transferred into the water phase with fast agitation. Once transfer was completed the mixture was neutralized with triethanolamine, followed by homogenization for 15 minutes. The mixture was cooled to 60° C. with continuous agitation, after which the water and sclerotium gum mixture was added to the mixture and further homogenized for an additional 15 minutes. Then bulk was then cooled to 50° C. and glyceryl polyacrylate, dimethiconol, and cyclomethicone were added to the batch, followed by further homogenization for 15 minutes. Then the remaining ingredients were added with mixing. Sodium hydroxide, kinetin, and water (1%) were premixed in a container, then added to the mixture. The mixture was then cooled to 30° C. and poured into suitable containers.

### EXAMPLE 3

A liquid foundation makeup formula was prepared as follows:

| INGREDIENT | w/w % |
|---|---|
| Water | QS |
| Glycerin | 1.00 |
| Butylene glycol | 1.00 |
| Cellulose gum | 0.10 |
| Magnesium aluminum silicate | 0.20 |
| Triethanolamine | 1.30 |
| Trisodium EDTA | 0.05 |
| Sorbitan sesquioleate | 0.20 |
| PEG-30 glyceryl cocoate | 1.00 |
| Oleth-3 phosphate | 0.10 |
| Ceteth-10 | 0.50 |
| Lecithin treated red iron oxide/talc | 0.38 |
| Lecithin treated iron oxides | 0.94 |
| Lecithin treated titanium dioxide | 3.00 |
| Lecithin treated talc | 3.97 |
| Mica, iron oxides, soy amino acids, | 0.20 |
| acacia dealbata wax | |
| Nylon-12 | 3.00 |
| Talc, soy amino acids, acacia dealbata wax | 3.50 |
| Micropulverized titanium dioxide | 1.00 |
| Titanium dioxide | 4.00 |
| Butylene glycol | 1.50 |
| Xanthan gum | 0.15 |
| Meadowfoam seed oil | 2.00 |
| Dimethicone | 10.00 |
| Isostearic acid | 3.50 |
| Propylene glycol dicaprylate/dicaprate | 5.60 |
| Isocetyl stearate | 3.40 |
| Phenyl trimethicone | 1.85 |
| Octinoxate | 3.50 |
| Glyceryl stearate/sodium lauryl sulfate | 0.50 |
| Propyl paraben | 0.10 |
| Steareth-2 | 0.75 |
| Zinc oxide/dimethicone | 0.20 |
| Glycerin | 2.00 |
| Butylene glycol | 2.50 |
| Talc, lecithin | 0.01 |
| Methyl paraben | 0.25 |
| Tocopherol | 0.40 |
| Retinyl palmitate | 0.08 |
| Methyldihydrojasmonate | 0.20 |
| Pectin | 0.05 |
| Methoxypropylgluconamide | 0.50 |
| Hydrolyzed wheat protein | 0.10 |
| Hydrolyzed glycosaminoglycans | 2.00 |
| Sodium hyaluronate, hydrolyzed glycosaminoglycans | 0.10 |
| Hexapeptide-10 | 1.00 |
| Green tea glycospheres | 0.20 |
| Imidazolidinyl urea | 0.20 |

The composition was prepared by combining the oil and water phase ingredients separately and emulsifying to mix.

### EXAMPLE 4

An anhydrous foundation makeup was prepared as follows:

| INGREDIENT | w/w % |
|---|---|
| Cyclomethicone | 18.21 |
| Dimethicone | 13.31 |
| Propyl parraben/laureth-7 (33%) | 1.70 |
| Boron nitride | 1.63 |
| Iron oxides, methicone | 2.76 |
| Titanium dioxide, alumina, methicone | 4.76 |
| Titanium dioxide, cyclomethicone, | 27.21 |
| PEG/PPG-18/18/dimethicone, polyglyceryl-6-ricinoleate, | |
| stearic acid, aluminum hydroxide | |
| Zinc oxide, cyclomethicone, PEG-10 dimethicone, dimethicone | 11.34 |
| Titanium dioxide, cylcomethicone, dimethicone copolyol, | 8.62 |
| triethoxycaprylylsilane | |
| Mica, methicone | 5.22 |
| Silica | 0.34 |
| Nylon-12 | 2.34 |
| Boron nitride | 1.56 |
| Hexapeptide-10 | 1.00 |

The composition was prepared by combining the ingredients and mixing well.

### EXAMPLE 5

Various cosmetic formulations were made according to the following formulas:

| | 1 (pressed | 2 (face | 3 (con- |
|---|---|---|---|
| Ingredient | powder) | powder) | cealer) |
| Boron nitride | 20.00 | 5.00 | 3.00 |
| Silica | 0.50 | | 5.28 |
| Silica, sodium hyaluronate | 0.50 | 0.50 | — |
| Methoxypropylgluconamide | 0.10 | 0.10 | 0.10 |
| Methylparaben | 0.20 | 0.20 | 0.10 |
| Diazolidinyl urea | 0.10 | 0.10 | — |
| Propyl paraben | 0.10 | 0.10 | — |
| Ethyl paraben | 0.15 | 0.15 | — |
| Bismuth oxychloride | 15.00 | 5.00 | — |
| Polyethylene | 11.00 | 3.00 | — |
| Zinc stearate | 9.00 | 5.00 | — |
| Talc, methicone, mineral oil | 24.35 | — | — |
| Mica, iron oxides, soy amino acids, acacia | 0.20 | 0.02 | 0.20 |
| dealbata flower wax | | | |
| Mica, methicone, mineral oil | 10.00 | 15.00 | — |
| Titanium dioxide, isopropyl titanium | 2.95 | — | 3.50 |
| triisostearate | | | |
| Hydrogenated olive oil | 0.10 | — | — |
| Dimethicone | QS | — | 7.84 |
| Hexapeptide-10 | 0.25 | 0.25 | 0.25 |
| Water, gingko biloba extract, ginseng root | 0.25 | 0.25 | — |
| extract, camellia sinensis leaf extract, | | | |
| centaurea cyanus flower extract, vitis | | | |
| vinefer (grape) seed extract | | | |
| Dimethicone, dimethiconol | 1.70 | — | — |
| Talc, methicone, mineral oil | 0.10 | — | — |
| Talc, lecithin | — | QS | 1.435 |
| Mica, barium sulfate, titanium dioxide | — | 1.00 | 0.44 |
| Iron oxides, isopropyl titanium | — | 4.14 | — |
| triisostearate | | | |
| Nylon-12 | — | 10.00 | 1.00 |
| Talc, soy amino acids, acacia | — | 8.00 | 2.54 |
| dealbata wax | | | |
| Lauroyl lysine | — | 6.00 | 2.20 |
| Aloe barbadensis leaf extract | — | 0.10 | 0.10 |
| Dimethicone, trimethylsiloxysilicate | — | 3.00 | 0.25 |
| Coco caprylate/caprate | — | 2.00 | — |
| Phenyl trimethicone | — | 1.50 | — |
| Hydrogenated olive oil unsaponifiables, | — | 0.80 | — |
| black currant fruit extract | | | |
| Tocopheryl acetate | — | 0.10 | 0.02 |
| Tocopherol | — | 0.10 | — |
| Tridecyl trimellitate | — | — | 2.15 |
| Neopentyl glycol Diethylhexanoate | — | — | 6.15 |
| Sorbitan trioleate | — | — | 0.50 |
| Pentahydrosqualene | — | — | 0.50 |
| Isopropyl isostearate | — | — | 9.67 |
| Cyclomethicone, trimethylsiloxysilicate | — | — | 5.00 |
| BHT | — | — | 0.10 |
| Myristyl myristate | — | — | 1.40 |
| Candelilla wax | — | — | 0.96 |
| Tribehenin | — | — | 6.30 |
| Hydrogenated coco-glycerides | — | — | 1.86 |
| Saxifraga sarmentosa extract, vitis | — | — | 0.02 |
| vinefera fruit extract, butylene glycol, | | | |
| water, morus bombycis root | | | |
| extract, scutellaria baicalensis | | | |
| root extract, disodium EDTA | | | |
| Salicylic acid, hydrolyzed vegetable | — | — | 0.02 |
| protein | | | |
| Titanium dioxide, aluminum hydroxide, | — | — | 22.00 |
| stearic acid, dimethicone, isopropyl | | | |
| isostearate | | | |
| Zinc oxide, dimethicone, isopropyl | — | — | 4.00 |
| isostearate | | | |
| Bismuth oxychloride | — | — | 2.00 |
| Titanium dioxide | — | — | 2.00 |
| Trimethylsiloxy silicate, cyclomethicone, | — | — | 4.07 |
| iron oxides | | | |
| Preservatives | — | — | 1.40 |
| Retinyl palmitate | — | — | 0.02 |
| Lauryl PEG/PPG-18/18 methicone | — | — | 0.50 |
| Magnesium ascorbyl phosphate | — | — | 0.02 |
| Polyglyceryl-4-isostearate | — | — | 1.00 |

The compositions were prepared by combining the ingredients and mixing well. Unless otherwise noted, all % values given herein are by weight % (i.e., wt. %).

### EXAMPLE 5

Skin cream formulas suitable for night skin treatment were made as follows:

| | % by weight | |
|---|---|---|
| Ingredients | Crème | Lotion |
| Water | QS | QS |
| Glycerine | 3.50 | 2.00 |
| Butylene glycol | 3.00 | 5.00 |
| Methyl paraben | 0.30 | 0.25 |
| Ethyl paraben | 0.20 | 0.20 |
| Trisodium EDTA | 0.10 | — |
| Disodium EDTA | — | 0.15 |
| Aloe barbadensis juice | 0.10 | — |
| Sodium citrate | — | 0.15 |
| Citric acid | — | 0.05 |
| Benzophenone-4 | — | 0.20 |
| Silica | 0.50 | 0.01 |
| Magnesium ascorbyl phosphate | 0.01 | — |
| Acrylates/C10-30 alkyl acrylate crosspolymer | 0.60 | 0.25 |
| HDI trimehylollactone crosspolymer | — | 0.50 |
| Caprylic/capric triglyceride | 6.50 | 4.00 |
| Glyceryl stearate, PEG-100 stearate | 3.50 | — |
| Polysorbate-20 | — | 0.20 |
| Sorbitan stearate | — | 0.20 |
| Ethylhexyl stearate | — | 7.00 |
| Stearic acid | — | 1.50 |
| Cetyl alcohol | — | 0.25 |
| Stearyl alcohol | — | 0.25 |
| Dimethicone | 1.00 | — |
| Neopentyl glycol diheptanoate | 6.50 | — |
| Sucrose polybehenate | 1.65 | — |
| Glyceryl stearate | 1.40 | — |
| Behenyl alcohol | 2.70 | — |
| Propyl paraben | 0.10 | — |
| BHT | — | 0.05 |
| Phenoxyethanol | — | 1.00 |
| Tocopheryl acetate | 0.10 | — |
| Retinyl palmitate | 0.01 | — |
| Triethanolamine | 1.00 | — |
| Tromethamine | — | 0.60 |
| Cyclomethicone | 1.00 | 1.00 |
| Cyclomethicone, dimethiconol | 1.80 | 1.00 |
| Bisabolol | 0.50 | 0.20 |
| Plukenetia Volubilis Seed Oil | 0.50 | 0.50 |
| Polyacrylamide, C13-14 isoparaffin, laureth-7 | — | 0.20 |
| Palmitoyl hydrolyzed wheat protein | 0.50 | 0.50 |
| Butyrospermum Parkii (shea butter) | — | 5.00 |
| Glyceryl polymethacrylate | 7.00 | — |
| Salicylic acid, hydrolyzed soy protein | 1.00 | 1.00 |
| Diazolidinyl urea | 0.30 | — |
| Potassium cetyl phosphate | — | 0.50 |
| Phytantriol | 0.10 | — |
| Biosaccharide Gum-1 | 1.00 | — |
| Water, glycerin, citrullas lanatus | 1.00 | — |
| (watermelon) fruit extract | | |
| Water, Hexapeptide-10 | 1.00 | 1.00 |
| Glycerin, water, sodium PCA, urea, trehalose, | 0.50 | — |
| polyquaternium-51, sodium hyaluronate | | |
| Hydrolyzed rice protein | 0.50 | 0.50 |
| Salicylic acid, hydrolyzed soy protein | — | 1.00 |
| Xanthan gum | — | 0.10 |
| Bisabolol | — | 0.20 |
| Water, Hibiscus Sabdariffa flower extract | — | 0.25 |
| FD&C Blue #1, 1% aqueous solution | 0.30 | — |
| Yellow 5 solution, 1% aqueous | 0.05 | — |
| Parfume | 0.50 | 0.50 |
| Mica, titanium dioxide | — | 0.50 |
| Red 4 solution, 0.5% | — | 0.10 |

The compositions were made by combining the oil and water phase ingredients separately, then emulsifying them together to form the compositions.

### EXAMPLE 5

Skin care compositions for day wear were made as follows:

| Ingredients | Lotion | Cream |
|---|---|---|
| Stearic acid | 2.75 | — |
| Cetyl alcohol | 0.45 | 0.75 |
| Stearyl alcohol | 0.25 | 0.50 |
| Glyceryl stearate | — | 1.00 |
| Polysorbate 20 | 0.20 | — |
| Sorbitan stearate | 0.20 | — |
| Octoxinate | 7.50 | 7.50 |
| Octisalate | 5.00 | 5.00 |
| Oxybenzone | 5.00 | 4.00 |
| Homosalate | — | 5.00 |
| Avobenzone | 2.00 | 3.00 |
| PVP/eicosene copolymer | 0.25 | 0.25 |
| BHT | 0.05 | — |
| Disodium EDTA | 0.15 | — |
| Trisodium EDTA | — | 0.15 |
| Sodium citrate | 0.15 | — |
| Magnesium ascorbyl phosphate | — | 0.001 |
| Citric acid | 0.06 | — |
| Butylene glycol | 4.00 | 5.00 |
| Ethyl paraben | 0.20 | 0.20 |
| Methyl paraben | 0.25 | 0.30 |
| Propyl paraben | — | 0.10 |
| Tocopheryl acetate | — | 0.10 |
| Tromethane | — | 0.25 |
| Xanthan gum | 0.25 | — |
| Potassium cetyl phosphate | 0.50 | 0.35 |
| Tromethane | 0.25 | — |
| Water | QS | — |
| HDI/trimethylol hexyllactone crosspolymer, silica | 0.50 | — |
| Cyclomethicone | 0.75 | 1.00 |
| Cyclomethicone, dimethiconol | 1.00 | — |
| Polyacrylamide, C13-14 isoparaffin, laureth-7 | 0.20 | — |
| Acrylates/C10-30 alkyl acrylate crosspolymer | — | 0.25 |
| Bisabolol | 0.20 | 0.50 |
| Plukenetia volubilis seed oil | 0.50 | 0.50 |
| Aloe barbadensis leaf extract | — | 0.10 |
| Palmitoyl hydrolyzed wheat protein | 0.50 | 0.50 |
| Phenethyl benzoate | — | 4.00 |
| Salicylic acid, hydrolyzed soy protein | 0.94 | 0.85 |
| Glycerin | 2.00 | — |
| Glycerin, water, sodium PCA, urea, trehelose, | — | 1.00 |
| polyquaternium-51, sodium hyaluronate | | |
| Water, Hibiscus sabdariffa flower extract | 0.25 | — |
| Hydrolyzed rice protein | 1.00 | 0.50 |
| Sodium hyaluronate, hydrolyzed glycosaminoglycans | — | 0.10 |
| Water, Hexapeptide-10 | 0.50 | 0.50 |
| Phenoxyethanol | 0.90 | 1.00 |
| Mica, titanium dioxide | — | 0.50 |
| Fragrance | 0.50 | 0.50 |

The compositions were prepared by combining the water and oil phase ingredients separately, then emulsifying together.

### EXAMPLE 6

| | % by weight | |
|---|---|---|
| Ingredient | Lip Cream | Eye Cream |
| Water | QS | QS |
| 1,2-hexanediol, capyryly glycol | 0.50 | — |
| Butylene glycol | — | 5.00 |
| Behenyl alcohol | — | 1.00 |
| Methyl paraben | 0.30 | 0.25 |
| Ethyl paraben | 0.20 | 0.15 |
| Xanthan gum | 0.20 | — |
| Hydroxyethyl cellulose | — | 0.05 |
| Propyl paraben | 0.10 | 0.10 |
| Magnesium ascorbyl phosphate | — | 0.001 |
| Potassium sorbate | — | 0.20 |
| Helianthus annus (sunflower) seed oil | 1.50 | — |
| Aloe barbadensis leaf extract | — | 0.10 |
| Persea gratissima (avocado) oil | 1.00 | — |
| PEG-8 beeswax | 2.50 | — |
| Cera Alba (beeswax) | 1.75 | — |
| Glyceryl stearate | 2.00 | — |
| Octyldodecyl neopentanoate | — | 3.50 |
| PPG-2 myristyl ether propionate | — | 1.00 |
| Steareth-2 | — | 1.50 |
| Steareth-21 | 0.75 | 1.85 |
| Cholesteryl/behenyl/dodecyl lauroyl glutamate | 0.02 | — |
| Tocopheryl acetate | 0.05 | 0.20 |
| Propylene glycol stearate | — | 1.50 |
| Ethylhexyl palmitate, triehenin, sorbitan | 0.50 | — |
| isostearate, palmitoyl oligopeptide | | |
| Mangifera Indica (Mango) seed butter | 2.75 | — |
| Butyrospermum parkii (shea butter) | 2.50 | — |
| Dimethicone | 1.00 | 2.50 |
| Neopentyl glycol diheptanoate | 1.85 | — |
| Silica dimethyl silylate | 0.50 | — |
| C10-30 cholesterol/lanosterol esters | 3.00 | — |
| Butylene glycol | 1.00 | — |
| HDI/trimethylol hexyllactone crosspolymer | 1.50 | — |
| Sorbitol | 3.00 | — |
| Bisabolol | 0.50 | 0.50 |
| Disodium EDTA | — | 0.10 |
| Plukenetia Volubilis seed oil | 0.50 | 0.50 |
| Palmitoyl hydrolyzed wheat protein | 0.50 | 0.50 |
| Acrylamide/sodium acryloyldimethyltaurate | 2.25 | — |
| copolymer, isohexadecane, polysorbate 80 | | |
| Water, Hexapeptide-10 | 0.50 | 1.00 |
| Glycerin, butylene glycol, water, carbomer, | 0.50 | — |
| polysorbate-20, palmitoyl oligopeptide, | | |
| palmitoyl tetrapeptide-3 | | |
| Salicylic acid, hydrolyzed soy protein | 0.10 | 0.85 |
| Microcrystalline wax | — | 0.75 |
| Diazolidinyl urea | 0.20 | — |
| Phenoxyethanol | — | 1.00 |
| Parfume | 0.10 | — |
| Butylene glycol dicaprylate/dicaprate | — | 2.00 |
| Nylon-12 | — | 0.50 |
| Polyacrylamide, C13-14 Isoparaffin, laureth-7 | — | 5.00 |
| Glycerin | — | 3.00 |
| Titanium dioxide | — | 0.15 |
| Panthenol | — | 0.10 |
| Sodium hyaluronate, hydrolyzed | — | 0.50 |
| glycosaminoglycans | | |
| Water, glycerin, hesperion, methyl chalcone, | — | 0.75 |
| steareth-20, dipeptide-2, | | |
| palmitoyl tetrapeptide-3 | | |
| Glycerin, steareth-20, chrysin, | — | 0.50 |
| n-hydroxysuccinate, palmitoyl oligopeptide, | | |
| palmitoyl tetrapeptide-3 | | |
| Water, Hypnia Musciformus extract, | — | 0.25 |
| Gellidiela Acerosea extract, | | |
| Cucumber Sativus seed extract | | |

A number of references have been cited, the entire disclosure of which are incorporated herein by reference.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A pigmented color cosmetic composition comprising a hexapeptide having Sequence ID No. 1, in a cosmetically acceptable carrier.

2. The composition of claim 1 wherein the cosmetically acceptable carrier is a foundation makeup, concealer, powder, eyeshadow, mascara, eyeliner, or blush.

3. The composition of claim 2, which is a water and oil emulsion foundation makeup or concealer composition.

4. The composition of claim 3 comprising from about 0.1-99% water, 0.1-99% oil, about 0.001-20% of one or more surfactants; and about 0.01-30% particulate material comprising pigments.

5. The composition of claim 4 wherein the oil comprises volatile silicones, volatile paraffinic hydrocarbons, non-volatile silicone oils, non-volatile esters, or mixtures thereof,

6. The composition of claim 5 wherein the volatile silicone oil is a linear volatile silicone selected from hexamethyldisiloxane, octamethyltrisiloxane, decamethyltetrasiloxane, dodecamethylpentasiloxane, or mixtures thereof.

7. The composition of claim 6 wherein the volatile paraffinic hydrocarbon is selected from isododecane, isohexadecane, or mixtures thereof.

8. The composition of claim 7 wherein the non-volatile silicone oil is dimethicone, phenyl trimethicone, phenyl dimethicone, or mixtures thereof.

9. The composition of claim 8 wherein the one or more surfactants are nonionic organic surfactants.

10. The composition of claim 9 wherein the nonionic organic surfactants are selected from alkoxylated alcohols; alkoxylated ethers; monomeric, homopolymeric, or copolymeric ethers; alkoxylated sorbitan; or polydimethylsiloxane polyoxyalkylene organosiloxane surfactants.

11. The composition of claim 10 wherein the nonionic organic surfactant is one or more alkoxylated alcohols.

12. The composition of claim 1 which is a foundation makeup comprising, by weight of the total composition:
about 0.1-99% water,
about 0.1-99% oil comprising at least one volatile oil selected from isododecane, isohexadecane, hexamethyldisiloxane, octamethyltrisiloxane, decamethyltetrasiloxane, dodecamethylpentasiloxane, decamethylcyclopentasiloxane, decamethylcyclohexasiloxane, and mixtures thereof; and at least one non-volatile oil comprising dimethicone, phenyl trimethicone, phenyl dimethicone, or esters,
about 0.001-20% of one or more nonionic organic surfactants selected from the group consisting of alkoxylated alcohols, alkoxylated ethers, and mixtures thereof,
about 0.01-30% particulate materials comprising pigments, powders, or mixtures thereof.

13. The composition of claim 12 wherein the volatile oil comprises decamethylcyclopentasiloxane.

14. The composition of claim 12 wherein the non-volatile oil comprises dimethicone, phenyl trimethicone, phenyl dimethicone, or mixtures thereof.

15. The composition of claim 12 wherein the nonionic organic surfactant comprises one or more alkoxylated alcohols.

16. The composition of claim 15 wherein the alkoxylated alcohol is a fatty alkoxylated alcohol.

17. The composition of claim 15 wherein the pigments are iron oxides.

18. The composition of claim 17 wherein the powders are bismuth oxychloride, mica, silica, boron nitride, talc, titanium dioxide, or mixtures thereof.

19. An emulsion skin care composition comprising at least one skin benefit agent in combination with a hexapeptide having Sequence ID No. 1 in a cosmetically acceptable carrier comprising a non-volatile dimethicone having viscosity ranging from about 5 to 1,000,000 centipoise at 25° C.

20. An emulsion skin care composition comprising at least one skin benefit agent and a hexapeptide having Sequence ID No. 1, in a cosmetically acceptable carrier comprising a UVA or UVB sunscreen or mixtures thereof.


SEQUENCE LISTING (1 sequence)

SEQ ID NO: 1 - protein, 6 aa, Artificial Sequence
  hexapeptide-10 (or Serilesine)
SIKVAV